(19) United States
(12) Patent Application Publication
(10) Pub. No.: US 2022/0362182 A1
(43) Pub. Date: Nov. 17, 2022

(54) METHODS FOR REVERSING HEPATIC STEATOSIS

(71) Applicant: BRIGHTSEED, INC., South San Francisco, CA (US)

(72) Inventors: Lee Heil CHAE, South San Francisco, CA (US); Fred LEVINE, La Jolla, CA (US)

(73) Assignee: SANFORD-BURNHAM MEDICAL RESEARCH INSTITUTE, La Jolla, CA (US)

(21) Appl. No.: 17/812,126

(22) Filed: Jul. 12, 2022

Related U.S. Application Data
(63) Continuation of application No. PCT/US2021/054570, filed on Oct. 12, 2021.
(60) Provisional application No. 63/091,766, filed on Oct. 14, 2020.

(51) Int. Cl. A61K 31/165; A61K 31/337; A61K 31/351; A61K 31/381; A61K 31/40; A61K 31/41; A61K 31/4409; A61K 31/277; A61K 31/167; A61K 31/4245; A61P 1/16

(52) U.S. Cl. CPC A61K 31/165; A61K 31/337; A61K 31/351; A61K 31/381; A61K 31/40; A61K 31/41; A61K 31/4409; A61K 31/277; A61K 31/167; A61K 31/4245; A61P 1/16

(57) ABSTRACT

Disclosed herein are methods for reversing hepatic steatosis by providing a consumable composition. Some embodiments provided include, for example, administering a compound of Formula (I) or compound of Formula (II). Some embodiments provide the composition is formulated as a dietary supplement, food ingredient or additive, a medical food, nutraceutical or pharmaceutical composition.

### BACKGROUND

Fatty liver disease is a major cause of morbidity and mortality. Excessive haptic fat storage secondary to obesity causes hepatocyte dysfunction, termed non-alcoholic fatty liver disease (NAFLD). NAFLD progresses in many cases to non-alcoholic steatotic hepatitis (NASH), characterized by inflammation, fibrosis, and hepatocyte death. In some individuals, this progresses further to cirrhosis and organ failure. Obesity-associated liver disease is a leading cause of liver transplantation.

A major challenge for treating lipotoxic diseases is identifying targets that affect fundamental aspects of disease pathogenesis. HNF4a is a highly attractive target as it plays a central role in controlling metabolism in the liver and the pancreatic b-cell, major players in the pathogenesis of NAFLD and T2D.

### SUMMARY OF THE DISCLOSURE

Disclosed herein are methods related to reversing hepatic steatosis. In some embodiments, a method for reversing hepatic steatosis comprises providing a consumable composition comprising at least one carrier and an effective amount of an extract comprising a compound of Formula (I), or an isomer, salt, homodimer, heterodimer, or conjugate thereof:

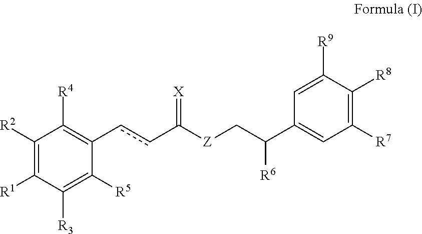

wherein

R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;

X is CH₂ or O;

Z is CHR^{a}, NR^{a}, or O; and

R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, thereby reversing hepatic steatosis.

Disclosed herein are methods for promoting fat clearance. In some embodiments, a method for promoting fat clearance comprises providing a consumable composition comprising at least one carrier and an effective amount of an extract comprising a compound of Formula (I), or an isomer, salt, homodimer, heterodimer, or conjugate thereof:

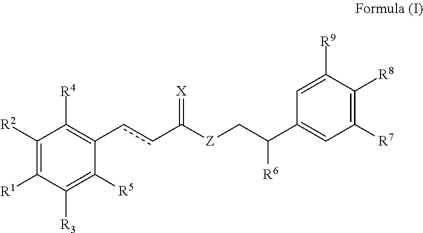

wherein

R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C -amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl;the dashed bond is present or absent;

X is CH₂ or O;

Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, thereby promoting fat clearance.

### BRIEF DESCRIPTION OF THE DRAWINGS

The features and advantages of the compositions and methods described herein will become apparent from the following description, taken in conjunction with the accompanying drawings. These drawings depict certain aspects of the compositions and methods described in the present application, and thus, are not to be considered limiting. In the drawings, similar reference numbers or symbols typically identify similar components, unless context dictates otherwise. The drawings may not be drawn to scale.

FIG. 1 illustrates the effects of N-trans-caffeoyltyramine (NCT) on DIO Mice. Diet-induced obese mice were injected IP twice per day with DMSO or N-trans-caffeoyltyramine (200 mg/kg/dose) for 14 days. Following sacrifice, organs were harvested, weighed, and processed for histology. N-trans-caffeoyltyramine decreased liver weight (A), but there was an increase in epididymal fat pad weight (B). Serum FFA were increased (C) and ALP was decreased (D). Each dot represents one mouse. *p<0.05, **p<0.01.

FIG. 2 illustrates N-trans-caffeoyltyramine (NCT) reverses hepatic steatosis in DIO mice. Diet-induced obese mice were injected IP twice per day with DMSO or N-trans-caffeoyltyramine (200 mg/kg/dose) for 14 days. Following sacrifice, livers were harvested from mice injected with DMSO (A-C) or N-trans-caffeoyltyramine (D-F) for staining with Oil Red O (all panels from independent mice) or for quantitation of Oil Red O staining (G) and triglyceride (TG) determination (H). N-trans-caffeoyltyramine significantly reduced the liver triglyceride level (p=0.007). *p<0.05, **p<0.01. n=12 mice per group.

FIG. 3 illustrates N-trans-caffeoyltyramine reverses the loss of HNF4a expression in the liver of DIO mice. Sections of liver from the mice from FIG. 1 were stained for Oil Red I and HNF4a (green nuclear staining), and DAPI (blue). N-trans-caffeoyltyramine significantly reduced the Oil Red O and increased the HNF4a staining.

FIG. 4 illustrates a model of the control of hepatic fat storage by HNF4α.

FIG. 5 illustrates an assay for HNF4α activity.

FIG. 6 illustrates results from insulin promoter, estrogenic, PPARγ agonist, and fat clearance assays. Panel A illustrates an assay for estrogenic activity. Panel B illustrates an assay for PPARγ agonist activity. Panel C illustrates a fat clearance assay.

FIG. 7 illustrates insulin and HNF4α mRNA assays measured by qPCR as an additional measure of HNF4α activity).

FIG. 8 illustrates the quantification of GFP-positive cells, reflecting activity of human insulin promoter-GFP transgene in T6PNE cells, which was performed with multiple doses of NCT and NFT to demonstrate dose-responsiveness using a Celigo imaging cytometer (N=14).

FIG. 9 illustrates the results of assays measuring insulin and HNF4α mRNA levels as measured by qPCT with multiple doses of NCT and NFT (N=4).

FIG. 10 illustrates an assay demonstrating HNF4α siRNA was blocked the effect of HNF4α agonists.

FIG. 11 illustrates representative images for data shown in FIG. 10.

FIG. 12 illustrates a DARTS assay to detect effect of compounds on HNF4α protease sensitivity. For the DARTS assay on the left, HepG2 cells were treated with DMSO (lane 1), BI6015 (lane 2), NCT (lane 3), or NFT (lane 4) at a concentration of 40 or 80 μM for 16 h. Total cell protein was extracted and each sample was split into two aliquots for proteolysis without (−) or with (+) subtilisin and analyzed by Western blotting for HNF4α. After detection of HNF4α, the membrane was stained with Ponceau S (magenta color) as a control to ensure that the compounds did not induce nonspecific proteolysis (Lane M has MW markers). All compounds were run on the same gel. For the HNF4α assay on the right, the HNF4α level was quantified by ImageJ using the western blots from the DARTS panel on the left.

FIG. 13 illustrates photomicrographs of representative wells stained for fat with Oil Red O (upper panels) or Nile Red (lower panels).

FIG. 14 illustrates the quantification of Nile Red staining that was done on a per cell basis using a Celigo imaging cytometer based on FIG. 13.

FIG. 15 illustrates an assay demonstrating triglyceride level was normalized to cellular protein measured by BCA and fold change was calculated relative to DMSO control (N=6).

FIG. 16 illustrates an assay validating of siRNAs. T6PNE cells were transfected with siRNA to each target gene. Two days later, cells were harvested for RNA isolation. QPCR was performed and normalized to the level of 18s rRNA. All siRNAs induced a significant decrease in the level of the target mRNA. Values represent the mean ±SE of 3 technical replicates, *p<0.05, (vs scrambled siRNA for each gene).

FIG. 17 illustrates an assay of candidate genes induced by NCT that have a role in fat metabolism screened for a role in NCT-induced fat clearance using siRNAs.

FIG. 18 illustrates the quantification of the Nile Red-positive cells processed in FIG. 17.

FIG. 19 illustrates pictographs demonstrating SPNS2 and S1PR3, but not SPHK2, are required for NCT-O.

FIG. 20 illustrates the quantification of the cellular fat detected by Nile Red staining as depicted in FIG. 19.

FIG. 21 illustrates photomicrographs of T6PNE cells treated for 2 days with 0.25 mM palmitate and the indicated compounds, followed by staining for fat with Nile Red.

FIG. 22 illustrates the quantification of Nile Red staining as described in FIG. 21.

FIG. 23 illustrates pictographs demonstrating inhibition by siRNA to SPNS2 or S1PR3 of fat clearance induced by DH-Cer. T6PNE cells were transfected with siRNAs to SPNS2 or S1PR3. Two days later, DH-Cer was added for 2 days, followed by staining with Oil Red.

FIG. 24 illustrates the quantification of number of cells positive for Nile Red from FIG. 23 demonstrating that DH-Cer-induced fat clearance requires SPNS2 and S1PR3.

FIG. 25 illustrates pictographs of DES-1 inhibitors GT-11 and B-0027 increase fat clearance. T6PNE cells were treated with 0.25 mM palmitate and DMSO or NCT.

FIG. 26 illustrates the quantification of the Nile Red cells shown in FIG. 25.

FIG. 27 illustrates an anti-LC3B Western blot demonstrating an increased ratio of LC3B II to LC3B I. For Western blotting, T6PNE cells were treated with DMSO (lane 1), NCT (10 μm), rapamycin (10 μM), and blotted with LC3B antibody. After detecting LC3B or p62, the same membrane was reblotted with anti-β-actin antibody to ensure equal amounts of protein in each lane.

FIG. 28 illustrates the quantification of ratio in LC3B II to LC3B I as depicted in FIG. 27.

FIG. 29 illustrates a p62 Western blot with T6PNE cells treated with NCT (10 μM), RA (10 μM), NCT+RA (each at 10 μM), NFT (10 μM), fenretinide (5 682 M), 4-OH-RA (20 μM), or without palmitate.

FIG. 30 illustrates the quantification of p62 protein expression normalized to actin as depicted in FIG. 29. The same membrane was reblotted with anti-β-actin antibody to ensure equal amounts of protein in each lane. Fenretinide had a statistically significant effect but retinoic acid did not.

FIG. 31 illustrates images of T6PNE cells treated for 2 days with or without palmitate, NCT (5 μM), fenretinide(5 μM), and the LAL inhibitor Lalistat2 (20 μM), followed by staining with Nile Red to visualize intracellular fat.

FIG. 32 illustrates quantification of the conditions shown in FIG. 31.

FIG. 33 illustrates Lalistat2 inhibits the effect of NCT on fat clearance. Cells from FIG. 31 were harvested for TG quantification, supporting the Nile Red staining shown in FIGS. 31 and 32.

FIG. 34 illustrates a DIO mouse (C57BL/6J) injected intraperitoneally with NCT (200 mg/kg bid) for two weeks followed by harvesting of organs with the box indicating the area of the liver, demonstrating a marked difference in color.

FIG. 35 illustrates a dissected lever from a DIO mouse indicating difference in color and weight.

FIG. 36 illustrates the quantified of the conditions described in FIG. 34, N=12 for each group.

FIG. 37 illustrates an assay for hepatic triglyceride (TG) content normalized to hepatic protein (Normal chow control, N=3, DMSO and NCT, N=12).

FIG. 38 illustrates a representative photomicrograph of hepatic Oil Red O staining (scale bar=200 μm).

FIG. 39 illustrates the Oil Red O quantification described in FIG. 38.

FIG. 40 illustrates an assay measuring the body weight at the initiation of the experiment (Day 0) and following 2 weeks of IP injection of DMSO and NCT (N=12 for each group).

FIG. 41 illustrates epididymal fat pads from representative mice showing increased weight with NCT (N=12 for each group).

FIG. 42 illustrates the quantified results described in FIG. 41.

FIG. 43 illustrates an assay for serum free fatty acid (FFA level) in DIO mice (Normal chow control, N=5, and DMSO and NCT, N=12).

FIG. 44 illustrates liver profiles on blood and serum TG level from mice injected with NCT.

FIG. 45 illustrates an assay determining blood alkaline phosphatase (ALP) level (Normal chow control, N=3 and DMSO and NCT, N=12).

FIG. 46 illustrates an assay for determination of the markers of liver function shown using a VetScan panel.

FIG. 47 illustrates HNF4a and CYP26a1 mRNA is induced by NCT in primary human hepatocytes. Human primary hepatocytes were seeded on Matrix with lean media (Day 0) and changed to high fat media plus DMSO or NCT (5, 15, 40 mM) on Day 4. At Day 10, cells were harvested for RNA extraction. HNF4a and CYP26a1, but not SPNS2 mRNAs were significantly induced by NCT. Values represent the mean ±SE of 3 biological replicates, *p<0.05 (vs DMSO).

FIG. 48 illustrates liver sections stained for Bodipy (green), HNF4α (red), DAPI (blue) and merged images in mice fed normal chow or HFD plus DMSO or NCT.

FIG. 49 illustrates the quantification of HNF4α nuclear staining as described in FIG. 48.

FIG. 50 illustrates the quantification of the hepatic HNF4a mRNA level as described in FIG. 48.

FIG. 51 illustrates SPNS2 mRNA was induced by NCT in T6PNE and mouse pancreas but not mouse liver. SPNS2 qPCR was performed on cDNA from T6PNE cells, mouse liver, and mouse pancreas. The Ct value for SPNS2 amplification in pancreas-derived samples was 31 for mouse pancreatic tissue but was 23 for mouse liver, reflecting a much higher level of expression. Values represent the mean ±SE of 6-9 biological replicates, *p<0.01(vs DMSO).

FIG. 52 illustrates RT-PCT analysis of hepatic CYP26A1 mRNA level (Normal chow control; N=5 and for DMSO and CNT; N=10).

FIG. 53 illustrates RT-PCT analysis of CYP26A1 mRNA level in T6PNE cells treated for 2 days with DMSO, NCT (10 μM), RA (10 μM), NCT+RA (10 μM), NFT (20 μM), fenretinide (5 μM), 4-OH-RA (20 μM) on 0.25 mM palmitate and DMSO w/o palmitate.

FIG. 54 illustrates T6PNE cells treated with palmitate (0.25 mM) plus the indicated compounds for 2 days, including the inhibitors ABT (10 mM, broad CYP inhibitor) and Talarozole (10 μM, selective CYP26 inhibitor).

FIG. 55 illustrates that quantification of the effect of CYP inhibitors on fat clearance by NCT (vs NCT for significance) from FIG. 54.

FIG. 56 illustrates representative images of T6PNE cells treated for 2 days with NCT or the RA metabolites 4-OXO-RA, 5,6-epoxy-RA, or 4-OH-RA (20 μM) and stained with Nile Red.

FIG. 57 illustrates the quantification of the effect of RA metabolites on fat clearance as described in FIG. 56.

FIG. 58 illustrates a line graph of T6PNE cells that were harvested 2 days after treatment with DMSO, NCT+RA (10 μM) and Fenretinide (5 μM). NCT and fenretinide induced multiple identical dihydroceramides.

FIG. 59 illustrates the ceramide (Cer)/dihydroceramide (DH-Cer) ratio was decreased in T6PNE cells treated with NCT+RA or Fenretinide.

FIG. 60 illustrates an assay from livers of mice treated with DMSO or NCT for 2 weeks.

### DETAILED DESCRIPTION

This disclosure provides, among other things, the discovery of strong HNF4α agonists and their use to uncover a previously unknown pathway by which HNF4α controls the level of fat storage in the liver. While not wishing to be bound by theory, it is believed that this involves the induction of lipophagy by dihydroceramides, the synthesis and secretion of which is controlled by genes induced by HNF4α. The HNF4α activators are N-transcaffeoyltyramine (NCT) and N-transferuloyltyramine (NFT), which are structurally related to known drugs alverine and benfluorex, which are weak HMF4α activators. With in vitro studies described herein, NCT and NFT induced fat clearance from palmitate-loaded cells. With in vivo assays described herein, in DIO mice, NCT led to recovery of normal hepatic HNF4α expression that is impaired by elevated levels of serum free fatty acids and reduction of steatosis. Mechanistically, increased dihydroceramide production and action downstream of HNF4α occurred through increased expression of HNF4α downstream genes, including SPNS2 and CYP26A1. In some embodiments, NCT was found to be completely nontoxic at the highest dose administered.

In aspects, compounds and compositions containing tyramine containing hydroxycinnamic acid amides are provided herein. Some embodiments provided herein provide for the compounds and compositions for the use in methods of promoting fat clearance and reversing hepatic steatosis.

### Compositions

In some aspects, the disclosure provided herein disclosure provides plant-derived aromatic metabolites with one or more acidic hydroxyl groups attached to aromatic arenes, and their use in modulating metabolism. In one embodiment, the plant-derived aromatic metabolite is a structural analog of compound 1:

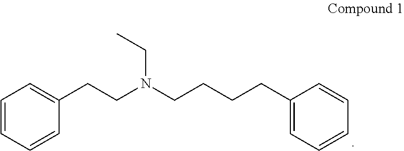 [SMILES: CCN(CCCCc1ccccc1)CCc1ccccc1]

In particular, the disclosure encompasses a compound of Formula (I), or an isomer, salt, homodimer, heterodimer, or conjugate thereof:

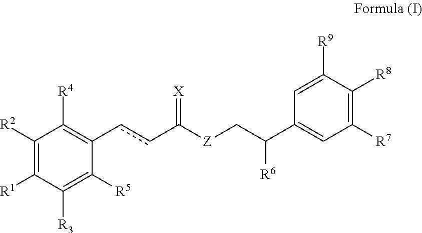

In some embodiments, R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, R², R³, and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, and R⁴, R⁵, R⁶, R⁷, and R⁹ are each independently hydrogen, deuterium, hydroxyl, or halogen;

In some embodiments, R¹, R², and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl, and R³, R⁴, R⁵, R⁶, R⁷, and R⁹ are each independently hydrogen, deuterium, hydroxyl, or halogen.

In some embodiments, the dashed bond is present or absent.

In some embodiments, X is CH₂ or O.

In some embodiments, Z is CHR^{a}, NR^{a}, or O.

In some embodiments, R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, a compound of Formula (I) is provided as a pharmaceutically acceptable salt or solvate thereof.

In some embodiments, a compound of Formula (I) is selected from (E)-3-(3,4-dihydroxyphenyl)-N-(4-ethoxyphenethyl)acrylamide, (E)-3-(3, 4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4sobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, 3-(3,4-dihydroxyphenyl)-N-(4-(methylsulfonamido)phenethyl)propanamide, or pharmaceutical salts, solvates, and combination of the foregoing.

In some embodiments, the disclosure encloses a compound of Formula (II):

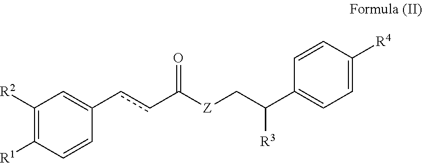

In some embodiments, R¹, R², R³, and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl

In some embodiments, the dashed bond is present or absent.

In some embodiments, Z is CHR^{a}, NR^{a}, or O.

In some embodiments, IV is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, a compound of Formula (II) is selected from (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acryl amide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-(4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4-dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenylhydrogen carbonate, (E)-3-(4-hydroxy-3-(pyridin-4-yloxy)phenyl)-N-(4-hydroxyphenethyl)acrylamide, (E)-3-(4-hydroxy-3-isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, 3-(3,4-dihydroxyphenyl)-N-(4-(methylsulfonamido)phenethyl)propanamide, or pharmaceutical salts, solvates, and combination of the foregoing.

In some embodiments, a compound of Formula (II) is provided as a pharmaceutically acceptable salt or solvate thereof.

In some embodiments, the disclosure encloses a compound of Formula (III):

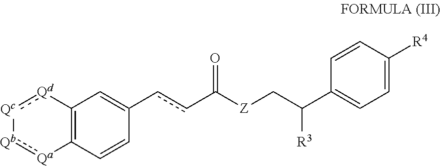

In some embodiments, R³ and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted — (O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₂₋₁₂heterocyclyl, optionally substituted —(O)C₅₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, the each independently selected dashed bond is present or absent.

In some embodiments, Z is CHR^{a}, NR^{a}, or O.

In some embodiments, Ra is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted —(O)C₄₋₁₂cycloalkyl, optionally substituted —(O)C₄₋₁₂heterocyclyl, optionally substituted —(O)C₄₋₁₂cycloalkyl, optionally substituted —(O)C₁₋₆alkylC₅₋₁₂aryl, optionally substituted —(O)C₁₋₆alkylC₅₋₁₂heteroaryl.

In some embodiments, Q^{a}, Q^{b}, Q^{c}, Q^{d} are each independently selected from a bond, CHR^{a}, NR^{a}, C═O, and —O—.

In some embodiments, Ra is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

In some embodiments, Q^{c}, Q^{d} are absent. In some embodiments, Q^{d} is absent.

In some embodiments, n is 1, 2, 3, or 4

In some embodiments, a compound of Formula (II) is provided as a pharmaceutically acceptable salt or solvate thereof.

“Isomer” refers to especially optical isomers (for example essentially pure enantiomers, essentially pure diastereomers, and mixtures thereof) as well as conformation isomers (i.e., isomers that differ only in their angles of at least one chemical bond), position isomers (particularly tautomers), and geometric isomers (e.g., cis-trans isomers).

In certain embodiments, a compound of Formula (I) or Formula (II) is selected from:

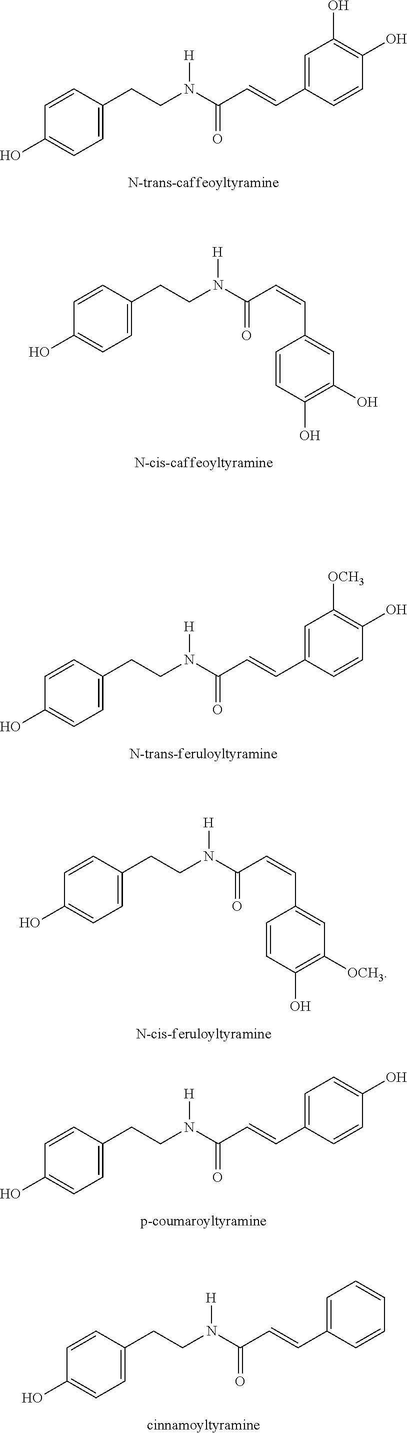 [SMILES: O=C(/C=C/c1ccc(O)c(O)c1)NCCc1ccc(O)cc1 ; O=C(/C=C\c1ccc(O)c(O)c1)NCCc1ccc(O)cc1 ; COc1cc(/C=C/C(=O)NCCc2ccc(O)cc2)ccc1O ; COc1cc(/C=C\C(=O)NCCc2ccc(O)cc2)ccc1O ; O=C(/C=C/c1ccc(O)cc1)NCCc1ccc(O)cc1 ; O=C(/C=C/c1ccccc1)NCCc1ccc(O)cc1]

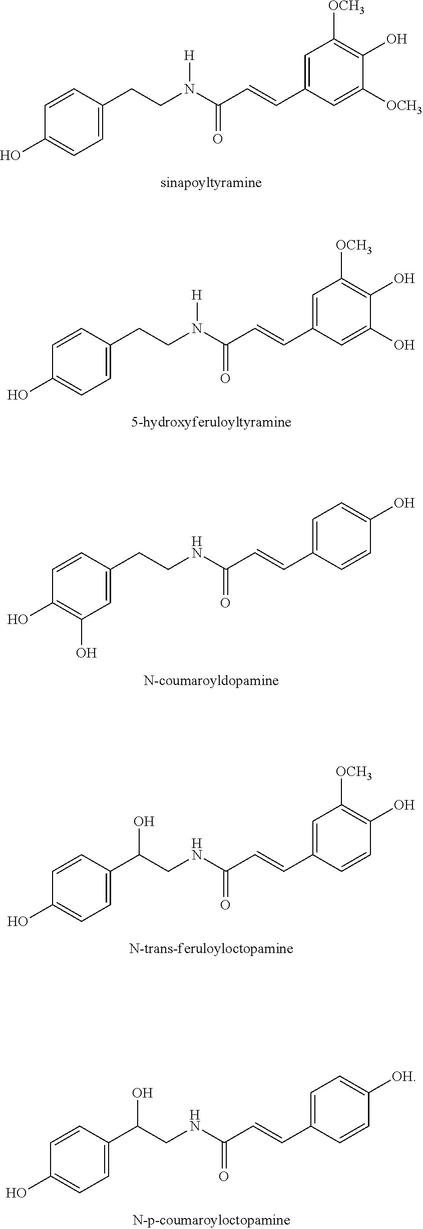 [SMILES: COc1cc(/C=C/C(=O)NCCc2ccc(O)cc2)cc(OC)c1O ; COc1cc(/C=C/C(=O)NCCc2ccc(O)cc2)cc(O)c1O ; O=C(/C=C/c1ccc(O)cc1)NCCc1ccc(O)c(O)c1 ; COc1cc(/C=C/C(=O)NCC(O)c2ccc(O)cc2)ccc1O ; O=C(/C=C/c1ccc(O)cc1)NCC(O)c1ccc(O)cc1]

A salt of a compound of this disclosure refers to a compound that possesses the desired pharmacological activity of the parent compound and includes: (1) an acid addition salt, formed with an inorganic acid such as hydrochloric acid, hydrobromic acid, sulfuric acid, nitric acid, phosphoric acid, and the like; or formed with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic camphorsulfonic acid, acid, 4-toluenesulfonic acid, 4-methylbicyclo[2.2.2]-oct-2-ene-1- carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) a salt formed when an acidic proton present in the parent compound is replaced.

As is known in the art, a homodimer is a molecule composed of two identical tyramine containing hydroxycinnamic acid amide subunits. By comparison, a heterodimer is a molecule composed of two different tyramine containing hydroxycinnamic acid amide subunits. Examples of homodimers of this disclosure include but are not limited to a cross-linked N-trans-feruloyltyramine dimer, a cross-linked N-trans-caffeoyl tyramine dimer and a cross-linked p-coumaroyltyramine dimer. See, for example, King & Calhoun (2005) Phytochemistry 66(20): 2468-73, which teaches the isolation of a cross-linked N-transferuloyltyramine dimer from potato common scab lesions.

Conjugates of monomers of tyramine containing hydroxycinnamic acid amide and other compounds, such as lignan amides. Examples of conjugates include, but are not limited to cannabisin A, cannabisin B, cannabisin C, cannabisin D, cannabisin E, cannabisin F and grossamide.

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents. If no substituents are indicated, it is meant that the indicated “optionally substituted” or “substituted” group may be individually and independently substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino, mono-substituted amino group and di-substituted amino group, and protected derivatives thereof.

For the groups herein, the following parenthetical subscripts further define the groups as follows: “(Cₙ)” defines the exact number (n) of carbon atoms in the group. For example, “C₁-C₆-alkyl” designates those alkyl groups having from 1 to 6 carbon atoms (e.g., 1, 2, 3, 4, 5, or 6, or any range derivable therein (e.g., 3-6 carbon atoms)).

In addition to isomers, salts, homodimers, heterodimers, and conjugates, the tyramine containing hydroxycinnamic acid amide may also be glycosylated. A glycosylated tyramine containing hydroxycinnamic acid amide may be produced by transglycosylating the tyramine containing hydroxycinnamic acid amide to add glucose units, for example, one, two, three, four, five, or more than five glucose units, to the tyramine containing hydroxycinnamic acid amide. Transglycosylation can be carried out with any suitable enzyme including, but not limited to, a pullulanase and isomaltase (Lobov, et al. (1991) Agric. Biol. Chem. 55:2959-2965), ˜-galactosidase (Kitahata, et al. (1989) Agric. Biol. Chem. 53:2923-2928), dextrine saccharase (Yamamoto, et al. (1994) Biosci. Biotech. Biochem. 58: 1657-1661) or cyclodextrin gluconotransferase, with pullulan, maltose, lactose, partially hydrolyzed starch and maltodextrin being donors.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, and hexyls. The alkyl group may be substituted or unsubstituted.

The term “halogen atom” or “halogen” as used herein, means any one of the radio-stable atoms of column 7 of the Periodic Table of the Elements, such as chloro (Cl), fluoro (F), bromo (Br) and iodo (I) groups.

In any of the groups described herein, an available hydrogen may be replaced with an alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, alkoxyalkoxy, alkoxycarbonyl, acyl, halo, nitro, aryloxycarbonyl, cyano, carboxy, aralkoxycarbonyl, alkyl sulfonyl, aryl sulfonyl, heteroarylsulfonyl, alkylthio, arylthio, heteroarylthio, aralkylthio, heteroaralkylthio, cycloalkyl, or heterocyclyl.

Any undefined valency on an atom of a structure shown in this application implicitly represents a hydrogen atom bonded to the atom.

As used herein, “alkenyl” refers to an alkyl group, as defined herein, that contains in the straight or branched hydrocarbon chain one or more double bonds. An alkenyl group may be unsubstituted or substituted.

As used herein, “alkynyl” refers to an alkyl group as defined herein, that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl, and cyclooctyl.

As used herein, “aryl” refers to a carbocyclic (all carbon) monocyclic or multicyclic aromatic ring system (including, e.g., fused, bridged, or spiro ring systems where two carbocyclic rings share a chemical bond, e.g., one or more aryl rings with one or more aryl or non-aryl rings) that has a fully delocalized pi-electron system throughout at least one of the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene, and azulene. An aryl group may be substituted or unsubstituted.

As used herein, “heterocyclyl” refers to mono- or polycyclic ring systems including at least one heteroatom (e.g., O, N, S). Such systems can be unsaturated, can include some unsaturation, or can contain some aromatic portion, or be all aromatic. A heterocyclyl group can contain from 3 to 30 atoms. A heterocyclyl group may be unsubstituted or substituted.

In particular embodiments, R¹ is present and represents a hydroxy group at the para position and R² is a hydroxy or lower alkoxy group at the meta position. In certain embodiments, the tyramine containing hydroxycinnamic acid amide having the structure of Formula (I) is in the trans configuration.

As used herein, “heteroaryl” refers to a monocyclic or multicyclic aromatic ring system (a ring system having a least one ring with a fully delocalized pi-electron system) that contain(s) one or more heteroatoms, that is, an element other than carbon, including but not limited to, nitrogen, oxygen, and sulfur, and at least one aromatic ring. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

The term “amino” as used herein refers to a —NH₂ group.

As used herein, the term “hydroxy” refers to a —OH group.

A “cyano” group refers to a “—CN” group.

A “carbonyl” group refers to a C=O group.

A “C-amido” group refers to a “—C(═O)N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. A C-amido may be substituted or unsubstituted.

An “N-amido” group refers to a “RC(═O)N(R_{A})—” group in which R and R_{A} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. An N-amido may be substituted or unsubstituted.

A “urea” group refers to a “—N(R_{A}R_{B})—C(═O)—N(R_{A}R_{B})-” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined above. A urea group may be substituted or unsubstituted.

The term “pharmaceutically acceptable salt” as used herein is a broad term, and is to be given its ordinary and customary meaning to a person of ordinary skill in the art (and is not to be limited to a special or customized meaning), and refers without limitation to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, and phosphoric acid. Pharmaceutical salts can also be obtained by reacting a compound with an organic acid such as aliphatic or aromatic carboxylic or sulfonic acids, for example formic acid, acetic acid (AcOH), propionic acid, glycolic acid, pyruvic acid, malonic acid, maleic acid, fumaric acid, trifluoroacetic acid (TFA), benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or naphthalenesulfonic acid. Pharmaceutical salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexylamine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, or may be stereoisomeric mixtures, and include all diastereomeric, and enantiomeric forms. In addition it is understood that, in any compound described herein having one or more double bond(s) generating geometrical isomers that can be defined as E or Z, each double bond may independently be E or Z a mixture thereof. Stereoisomers are obtained, if desired, by methods such as, stereoselective synthesis and/or the separation of stereoisomers by chiral chromatographic columns.

Likewise, it is understood that, in any compound described, all tautomeric forms are also intended to be included.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the compounds described herein can be labeled isotopically or by another other means, including, but not limited to, the use of chromophores or fluorescent moieties, bioluminescent labels, or chemiluminescent labels. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the methods and formulations described herein include the use of crystalline forms, amorphous phases, and/or pharmaceutically acceptable salts, solvates, hydrates, and conformers of compounds of some embodiments, as well as metabolites and active metabolites of these compounds having the same type of activity. A conformer is a structure that is a conformational isomer. Conformational isomerism is the phenomenon of molecules with the same structural formula but different conformations (conformers) of atoms about a rotating bond. In specific embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein. Other forms in which the compounds of some embodiments can be provided include amorphous forms, milled forms and nano-particulate forms.

Likewise, it is understood that the compounds described herein, such as compounds of some embodiments, include the compound in any of the forms described herein (e.g., pharmaceutically acceptable salts, prodrugs, crystalline forms, amorphous form, solvated forms, enantiomeric forms, tautomeric forms, and the like). Formulations

A substantially pure compound or extract comprising a compound of this disclosure can be combined with a carrier and provided in any suitable form for consumption by or administration to a subject. In this respect, the compound or extract is added as an exogenous ingredient or additive to the consumable. Suitable consumable forms include, but are not limited to, a dietary supplement, food ingredient or additive, a medical food, nutraceutical or pharmaceutical composition. In some embodiments, the compound or extract is provided in either a liquid or powder form.

A food ingredient or additive is an edible substance intended to result, directly or indirectly, in its becoming a component or otherwise affecting the characteristic of any food (including any substance intended for use in producing, manufacturing, packing, processing, preparing, treating, packaging, transporting, or holding food). A food product, in particular a functional food, is a food fortified or enriched during processing to include additional complementary nutrients and/or beneficial ingredients. A food product according to this disclosure can, e.g., be in the form of butter, margarine, sweet or savory spreads, condiment, biscuits, health bar, bread, cake, cereal, candy, confectionery, soup, milk, yogurt or a fermented milk product, cheese, juice-based and vegetable-based beverages, fermented beverages, shakes, flavored waters, tea, oil, or any other suitable food. In some embodiments, the food product is a whole-food product in which the concentration of the compound has been enriched through particular post-harvest and food production processing methods to levels that provide an efficacious amount of the compound.

A dietary supplement is a product taken by mouth that contains a compound or extract of the disclosure and is intended to supplement the diet. A nutraceutical is a product derived from a food source that provides extra health benefits, in addition to the basic nutritional value found in the food. A pharmaceutical composition is defined as any component of a drug product intended to furnish pharmacological activity or other direct effect in the diagnosis, cure, mitigation, treatment, or prevention of disease, or to affect the structure or any function of the body of humans or other animals. Dietary supplements, nutraceuticals and pharmaceutical compositions can be found in many capsules, forms such as tablets, coated tablets, pills, capsules, pellets, granules, softgels, gelcaps, liquids, powders, emulsions, suspensions, elixirs, syrup, and any other form suitable for use.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound described herein, including a compound of Formula (I), (II), (III), or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical excipient may also be prepared, placed in an appropriate container, and labeled for treatment of an indicated condition.

The compounds, salt and/or pharmaceutical composition can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. The kit can also contain separate doses of a compound(s) or pharmaceutical composition for serial or sequential administration. The kit can optionally contain one or more diagnostic tools and instructions for use. The kit can contain suitable delivery devices, for example., syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

In some embodiments, a compound of Formula (I), Formula (II), or Formula (III) is administered at a dose in the range of about 0.1 - 200 mg/kg body weight. In some embodiments, a compound of Formula (I), Formula (II), or Formula (III) is administered at a dose in the range of about 0.1-1, 0.5-1, 0.1-10, 0.5-10, 1-10, 1-20, 1-30, 1-40, 1-50, 1-60, 1-70, 1-80, 1-90, 1-100, 1-200, 1-300, 1-400, 1-500, 1-600, 1-700, 1-800, 1-900, 1-1000, 1-11, 1-12, 1-13, 1-13, 1-14, 1-15, 1-16, 1-17, 1-18, 1-19, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 20-30, 20-40, 20-50, 20-60, 20-70, 20-80, 20-90, 20-100, 20-200, 20-300, 20-400, 20-500, 20-600, 20-700, 20-800, 20-900, 20-1000, 30-40, 30-50, 30-60, 30-70, 30-80, 30-90, 30-100, 30-200, 30-300, 30-400, 30-500, 30-600, 30-700, 30-800, 30-900, 30-1000, 40-50, 40-60, 40-70, 40-80, 40-90, 40-100, 40-200, 40-300, 40-400, 40-500, 40-600, 40-700, 40-800, 40-900, 40-1000, 50-60, 50-70, 50-80, 50-90, 50-100, 50-200, 50-300, 50-400, 50-500, 50-600, 50-700, 50-800, 50-900, 60-70, 60-80, 60-90, 60-100, 60-200, 60-300, 60-400, 60-500, 60-600, 60-700, 60-800, 60-900, 60-1000, 70-80, 70-90, 70-100, 70-200, 70-300, 70-400, 70-500, 70-600, 70-700, 70-800, 70-900, 70-1000, 80-90, 80-100, 80-200, 80-300, 80-400, 80-500, 80-600, 80-700, 80-800, 80-900, 80-100, 90-100, 90-200, 90-300, 90-400, 90-500, 90-600, 90-700, 90-800, 90-900, 90-1000, 100-150, 100-200, 100-300, 100-400, 100-500, 100-600, 100-700, 100-800, 100-900, or 100-1000 mg/kg of body weight. In some embodiments, a compound of Formula (I), Formula (II), or Formula (III) is administered at a dose of about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 80, 90, or 95 mg/kg of the body weight. In some embodiments, a compound of Formula (I), Formula (II), or Formula (III) is administered at a dose less than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, a compound of Formula (I), Formula (II), or Formula (III) is administered at a dose greater than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 mg/kg of a subjects body weight.

In some embodiments, a compound of Formula (I), Formula (II), or Formula (III) dose is about 0.1 mg-10 mg, 0.1 mg-25 mg, 0.1 mg-30 mg, 0.1 mg-50 mg, 0.1 mg-75 mg, 0.1 mg-100 mg, 0.5 mg-10 mg, 0.5 mg-25 mg, 0.5 mg-30 mg, 0.5 mg-50 mg, 0.5 mg-75 mg, 0.5 mg-100 mg, 1 mg-10 mg, 1 mg-25 mg, 1 mg-30 mg, 1 mg-50 mg, 1 mg-75 mg, 1 mg-100 mg, 2 mg-10 mg, 2 mg-25 mg, 2 mg-30 mg, 2 mg-50 mg, 2 mg-75 mg, 2 mg-100 mg, 3 mg-10 mg, 3 mg-25 mg, 3 mg-30 mg, 3 mg-50 mg, 3 mg-75 mg, 3 mg-100 mg, 4 mg-100 mg, 5 mg-10 mg, 5 mg-25 mg, 5 mg-30 mg, 5 mg-50 mg, 5 mg-75 mg, 5 mg-300 mg, 5 mg-200 mg, 7.5 mg-15 mg, 7.5 mg-25 mg, 7.5 mg-30 mg, 7.5 mg-50 mg, 7.5 mg-75 mg, 7.5 mg-100 mg, 7.5 mg-200 mg, 10 mg-20 mg, 10 mg-25 mg, 10 mg -50 mg, 10 mg-75 mg, 10 mg-100 mg, 15 mg-30 mg, 15 mg-50 mg, 15 mg-100 mg, 20 mg-20 mg, 20 mg-100 mg, 30 mg-100 mg, 40 mg-100 mg, 10 mg-80 mg, 15 mg-80 mg, 20 mg-80 mg, 30 mg-80 mg, 40 mg-80 mg, 10 mg-60 mg, 15 mg-60 mg, 20 mg-60 mg, 30 mg-60 mg, or about 40 mg-60 mg. In some embodiments, a compound of Formula (I), Formula (II), or Formula (III) administered is about 20 mg-60 mg, 27 mg-60 mg, 20 mg-45 mg, or 27 mg-45 mg. In some embodiments, a compound of Formula (I), Formula (II), or Formula (III) administered is about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 3 Omg-32 mg, 30 mg-34 mg, 30 mg-36 mg, 30 mg-38 mg, 30 mg-40 mg, 30 mg-42 mg, 30 mg-44 mg, 30 mg-46 mg, 3 Omg-48 mg, 30 mg-50 mg, 3 Omg-52 mg, 3 Omg-54 mg, 30 mg-56 mg, 30 mg-58 mg, 3 Omg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, a compound of Formula (I), Formula (II), or Formula (III) dose is greater than, equal to, or about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 300 mg. about 400 mg, about 500 mg, about 600 mg, about 700 mg, about 800 mg, about 900 mg, or about 1000 mg. In some embodiments, a compound of Formula (I), Formula (II), or Formula (III) dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

The term “carrier” as used herein means a material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier should be compatible with the other ingredients of the formulation and not injurious to the subject. Some examples of materials that can serve as carriers include: (1) sugars, such as lactose, glucose and sucrose; (2) starches, such as corn starch and potato starch; (3) cellulose, and its derivatives, such as sodium carboxymethyl cellulose, ethyl cellulose, cellulose acetate, and hydroxyl propyl methyl cellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/ or polyanhydrides; and (22) other nontoxic compatible substances employed in conventional formulations.

For preparing solid compositions such as tablets or capsules, the compound or extract is mixed with a carrier (e.g., conventional tableting ingredients such as corn starch, lactose, sucrose, sorbitol, talc, stearic acid, magnesium stearate, dicalcium phosphate or gums) and other diluents (e.g., water) to form a solid composition. This solid composition is then subdivided into unit dosage forms containing an effective amount of the compound of the present disclosure. The tablets or pills containing the compound or extract can be coated or otherwise compounded to provide a dosage form affording the advantage of prolonged action.

In particular embodiments of this disclosure, a consumable composition includes the compound or extract, a carrier and a preservative to reduce or retard microbial growth. The preservative is added in amounts up to about 5%, preferably from about 0.01% to 1% by weight of the film. Preferred preservatives include sodium benzoate, methyl parabens, propyl parabens, sodium nitrite, sulphur dioxide, sodium sorbate and potassium sorbate. Other suitable preservatives include, but are not limited to, salts of edetate, (also known as salts of ethylenediaminetetraacetic acid, or EDTA, such a disodium EDTA).

The liquid forms in which the compound or extract of the disclosure is incorporated for oral or parenteral administration include aqueous solution, suitably flavored syrups, aqueous or oil suspensions, and flavored emulsions with edible oils as well as elixirs and similar vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

Methods of preparing formulations or compositions of this disclosure include the step of bringing into association a compound or extract of the present disclosure with the carrier and, optionally, one or more accessory and/or active ingredients. In general, the formulations are prepared by uniformly and intimately bringing into association a compound or extract of the present disclosure with liquid carriers, or finely divided solid carriers, or both, and then, if necessary, shaping the product. As such, the disclosed formulation may consist of, or consist essentially of a compound or extract described herein in combination with a suitable carrier.

When a compound or extract of the present disclosure is administered as pharmaceuticals, nutraceuticals, or dietary supplements to humans and animals, they can be given per se or as a composition containing, for example, 0.1 to 99% active ingredient in combination with an acceptable carrier. In some embodiments, the compound or extract of the present disclosure may be administered at about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99% w/w, or ranges including and/or spanning the aforementioned values.

A consumable product may be consumed by a subject to provide less than 100 mg of a compound disclosed herein per day. In certain embodiments, the consumable provides between 10 and 60 mg/day of a tyramine containing hydroxycinnamic acid amide. The effective amount can be established by methods known in the art and be dependent upon bioavailability, toxicity, etc.

While it is contemplated that individual tyramine containing hydroxycinnamic acid amides may be used in the consumables of this disclosure, it is further contemplated that two or more of the compounds or extracts could be combined in any relative amounts to produce custom combinations of ingredients containing two or more tyramine containing hydroxycinnamic acid amides in desired ratios to enhance product efficacy, improve organoleptic properties or some other measure of quality important to the ultimate use of the product.

### Combinations

Some aspects relate to a combination of a compound of Formula (I), (II), or (III) with one or more compounds selected from a dihydrosphingosine, ceramide, glycosphingolipid, and a sphingosine. In some embodiments, the combination includes more compounds selected from dihydroceramide, ceramide, or a sphingosine.

In some embodiments, the ceramide is selected from the group consisting of natural ceramide, synthetic ceramide, a ceramide phosphate, a 1O-acyl-ceramide, a dihydroceramide, a dihydroceramide phosphate, and a 2-hydroxy ceramide.

In some embodiments, the natural ceramide is porcine brain or egg.

In some embodiments, he synthetic ceramide is selected from the group consisting of N-octadecanoyl-D-erythro-sphingosine (C18), N-hexadecanoyl-D-erythro-sphingosine (C16) N-acetoyl-D-erythro-sphingosine (C2 Ceramide, d18:1/2:0), N-butyroyl-D-erythro-sphingosine (C4 Ceramide, d18:1/4:0), N-hexanoyl-D-erythro-sphingosine (C6 Ceramide, d18:1/6:0), N-octanoyl-D-erythro-sphingosine (C8 Ceramide, d18:1/8:0), N-decanoyl-D-erythro-sphingosine (C10 Ceramide, d18:1/10:0), N-lauroyl-D-erythro-sphingosine (C12 Ceramide, d18:1/12:0), N-myristoyl-D-erythro-sphingosine (C14 Ceramide, d18:1/14:0), N-palmitoyl-D-erythro-sphingosine (C16 Ceramide, d18:1/16:0), N-heptadecanoyl-D-erythro-sphingosine (C17 Ceramide, d18:1/17:0), N-stearoyl-D-erythro-sphingosine (C18 Ceramide, d18:1/18:0), N-oleoyl-D-erythro-sphingosine (C18:1 Ceramide, d18:1/18:1(9Z)), N-arachidoyl-D-erythro-sphingosine (C20 Ceramide, d18:1/20:0), N-behenoyl-D-erythro-sphingosine (C22 Ceramide, d18:1/22:0), N-lignoceroyl-D-erythro-sphingosine (C24 Ceramide, d18:1/24:0), N-nervonoyl-D-erythro-sphingosine (C24:1 Ceramide, d18:1/24:1(15Z)), N-acetoyl-D-erythro-sphingosine (C17 base) (C2 Ceramide, d17:1/2:0), N-octanoyl-D-erythro-sphingosine (C17 base) (C8 Ceramide, d17:1/8:0), N-stearoyl-D-erythro-sphingosine (C17 base) (C₁₈ Ceramide, d17:1/18:0), N-oleoyl -D-erythro-sphingosine (C17 base) (C18:1 Ceramide, d17:1/18:1(9Z)), N-arachidoyl-D-erythro-sphingosine (C17 base) (C20 Ceramide, d17:1/20:0), N-lignoceroyl-D-erythro-sphingosine (C17 base) (C24 Ceramide, d17:1/24:0), and N-nervonoyl-D-erythro-sphingosine (C17 base) (C24:1 Ceramide, d17:1/24:1(15Z)).

In some embodiments, the ceramide phosphate is selected from the group consisting of N-acetoyl-ceramide-1-phosphate (ammonium salt) (C2 Ceramide-1-Phosphate, d18:1/2:0), N-octanoyl-ceramide-1-phosphate (ammonium salt) (C8 Ceramide-1-Phosphate, d18:1/8:0), N-lauroyl-ceramide-1-phosphate (ammonium salt) (C12 Ceramide-1-Phosphate, d18:1/12:0), N-palmitoyl-ceramide-1-phosphate (ammonium salt) (C16 Ceramide-1-Phosphate, d18:1/16:0), N-oleoyl-ceramide-1-phosphate (ammonium salt) (C18:1 Ceramide-1-Phosphate, d18:1/18:1(9Z)), N-lignoceroyl-ceramide-1-phosphate (ammonium salt) (C24 Ceramide-1-Phosphate, 18:1/24:0), N-acetoyl-ceramide-1-phosphate (C17 base) (ammonium salt) (C2 Ceramide-1-Phosphate, d17:1/2:0), and N-octanoyl-ceramide-1-phosphate (C17 base) (ammonium salt) (C8 Ceramide-1-Phosphate, d17:1/8:0).

In some embodiments, the dihydroceramide is selected from the group consisting of N-hexanoyl-D-erythro-sphinganine (C6 Dihydroceramide, d18:0/6:0), N-octanoyl-D-erythro-sphinganine (C8 Dihydroceramide, d28:0/8:0), N-palmitoyl-D-erythro-sphinganine (C16 Dihydroceramide, d18:0/16:0), N-stearoyl-D-erythro-sphinganine (C18 Dihydroceramide, d18:0/18:0), N-oleoyl-D-erythro-sphinganine (C18:1 Dihydroceramide, d18:0/18:1(9Z)), N-lignoceroyl-D-erythro-sphinganine (C24 Dihydroceramide, d28:0/24:0), and N-nervonoyl-D-erythro-sphinganine-D-erythro-sphinganine (C24:1 Dihydroceramide, d18:0/24:1(15Z)).

In some embodiments, the dihydroceramide phosphate is N-palmitoyl-D-erythro-dihydroceramide-1-phosphate (ammonium salt) (C16 Dihydroceramide-1-Phosphate, d18:0/16:0) or N-lignoceroyl-D-erythro-dihydroceramide-1-phosphate (ammonium salt) (C24 Dihydroceramide-1-Phosphate, d18:0/24:0).

In some embodiments, the 2-hydroxy ceramide is selected from the group consisting of N-(2′-(R)-hydroxyl auroyl)-D-erythro-sphingosine (12:0(2R—OH) Ceramide), N-(2′-(S)-hydroxylauroyl)-D-erythro-sphingosine (12:0(2S—OH) Ceramide), N-(2′-(R)-hydroxypalmitoyl)-D-erythro-sphingosine (16:0(2R—OH) Ceramide), N-(2′-(S)-hydroxypalmitoyl)-D-erythro-sphingosine (16:0(2S—OH) Ceramide), N-(2′-(R)-hydroxyheptadecanoyl)-D-erythro-sphingosine (17:0(2R—OH) Ceramide), N-(2′-(S)-hydroxyheptadecanoyl)-D-erythro-sphingosine (17:0(2S—OH) Ceramide), N-(2′-(R)-hydroxystearoyl)-D-erythro-sphingosine (18:0(2R—OH) Ceramide), N-(2′-(S)-hydroxystearoyl)-D-erythro-sphingosine (18:0(2S—OH) Ceramide), N-(2′-(R)-hydroxyoleoyl)-D-erythro-sphingosine (18:1(2R—OH) Ceramide), N-(2′-(S)-hydroxyoleoyl)-D-erythro-sphingosine (18:1(2S—OH) Ceramide), N-(2′-(R)-hydroxy arachidoyl)-D-erythro-sphingosine (20:0(2R—OH) Ceramide), N-(2′-(S)-hydroxylarachidoyl)-D-erythro-sphingosine (20:0(2S—OH) Ceramide), N-(2′-(R)-hydroxybehenoyl)-D-erythro-sphingosine (22:0(2R—OH) Ceramide), N-(2′-(S)-hydroxylbehenoyl)-D-erythro-sphingosine (22:0(2S—OH) Ceramide), N-(2′-(R)-hydroxylignoceroyl)-D-erythro-sphingosine (24:0(2R—OH) Ceramide), N-(2′-(S)-hydroxyllignoceroyl)-D-erythro-sphingosine (24:0(2S—OH) Ceramide), N-(2′-(R)-hydroxynervonoyl)-D-erythro-sphingosine (24:1(2R—OH) Ceramide), and N-(2′-(S)-hydroxylnervonoyl)-D-erythro-sphingosine (24:1(2S—OH) Ceramide).

In some embodiments, the sphingosine is selected from the group consisting of natural sphingosine, synthetic sphingosine, phosphorylated sphingosine (S1P), and methylated sphingosine.

In some embodiments, the natural sphingosine is D-erythro-sphingosine.

In some embodiments, the synthetic sphingosine is selected from the group consisting of sphingosine (d18:1), sphingosine (d17:1), sphingosine (d20:1), L-threo-sphingosine (d18:1), 1-deoxysphingosine, and 1-desoxymethylsphingosine. In some embodiments, the sphinganine is selected from the group consisting of sphinganine (d18:0), sphinganine (d17:0), sphinganine (d20:0), 1-deoxysphinganine, 1-desoxymethylsphinganine, and L-threo-dihydrosphingosine (d18:0) (Safingol). In some embodiments, the phosphorylated sphingosine is selected from the group consisting of sphingosine-1 -phosphate (d18:1), sphingosine-1-phosphate (DMA Adduct), sphingosine-1-phosphate (d17:1), sphingosine-1-phosphate (d20:1), sphinganine-1-phosphate (d18:0), sphinganine-1-phosphate (d17:0), and sphinganine-1-phosphate (d20:0). In some embodiments, the methylated sphingosine is selected from the group consisting of monomethyl sphingosine (d18:1), dimethyl sphingosine (d18:1), dimethyl sphingosine (d17:1), trimethyl sphingosine (d18:1), trimethyl sphingosine (d17:1), dimethyl sphinganine (d18:0), trimethyl sphinganine (d18:0), dimethyl sphingosine-1-phosphate (d18:1), and dimethyl sphinganine-1-phosphate (d18:0).

In some embodiments, the glycosphingolipid is selected from the group consisting of a natural glycosphingolipid, a glycosyl sphingolipid, a galactosyl sphingolipid, a lactosyl sphingolipid, a sulfatide, and a-galactosyl ceramide (αGalCer).

In some embodiments, the natural glycosphingolipid is selected from the group consisting of a cerebroside (e.g., from porcine brain), a glucocerebroside (e.g., from soy), a sulfatide (ammonium salt) (e.g., from porcine brain), a GM1 ganglioside (ammonium salt) (e.g., from ovine brain), a ganglioside GM1 (e.g., from ovine brain), and a total ganglioside extract (ammonium salt) (e.g., from porcine brain).

In some embodiments, the glycosyl sphingolipid is selected from the group consisting of D-glucosyl-β1′-D-erythro-sphingosine (Glucosyl(β) Sphingosine, d18:1), D-glucosyl-β1,1′N-octanoyl-D-erythro-sphingosine (C8 Glucosyl(β) Ceramide, d18:1/8:0), D-glucosyl-β1,1′N-lauroyl-D-erythro-sphingosine (C12 Glucosyl(β) Ceramide, d18:1/12:0), D-glucosyl-β1,1′N-palmitoyl-D-erythro-sphingosine (C16 Glucosyl(β) Ceramide, d18:1/16:0), D-glucosyl-β1,1′N-stearoyl-D-erythro-sphingosine (C18 Glucosyl(β) Ceramide, d18:1/18:0), D-glucosyl-β1,1′N-oleoyl-D-erythro-sphingosine (C18:1 Glucosyl(β) Ceramide, d18:1/18:1(9Z)), and D-glucosyl-β1′-N-nervonoyl-D-erythro-sphingosine (C_{24:1} Glucosyl(β) Ceramide, d18:1/24:1(15Z)).

In some embodiments, the galactosyl sphingolipid is selected from the group consisting of D-galactosyl-β1′-D-erythro-sphingosine (Galactosyl(β) Sphingosine, d18:1), N,N-dimethyl-D-galactosylβ1′-D-erythro-sphingosine (Galactosyl(β) Dimethyl Sphingosine, d18:1), D-galactosyl-β1′ N-octanoyl-D-erythro-sphingosine (C8 Galactosyl(β) Ceramide, d18:1/8:0), D-galactosyl-β1′ N-lauroyl-D-erythro-sphingosine (C12 Galactosyl(β) Ceramide, d18:1/12:0), D-galactosyl-β1′ N-palmitoyl-D-erythro-sphingosine (C16 Galactosyl(β) Ceramide, d18:1/16:0), and D-galactosyl-β1′N-nervonoyl-D-erythro-sphingosine (C_{24:1} Galactosyl(β) Ceramide, d18:1/24:1(15Z)).

In some embodiments, the lactosyl sphingolipid is selected from the group consisting of D-lactosyl-β1′-D-erythro-sphingosine (Lactosyl(β) Sphingosine, d18:1), D-lactosyl-β1′ N-octanoyl-D-erythro-sphingosine (C8 Lactosyl(β) Ceramide, d18:1/8:0), D-lactosyl-β1′-N-octanoyl-L-threo-sphingosine (C8 L-threo-Lactosyl(β) Ceramide, d18:1/8:0), D-lactosyl-β1′ N-lauroyl-D-erythro-sphingosine (C12 Lactosyl(β) Ceramide, d18:1/12:0), D-lactosyl-β1,1∝N-palmitoyl-D-erythro-sphingosine (C16 Lactosyl(β) Ceramide, d18:1/16:0), D-lactosyl-β1′N-lignoceroyl-D-erythro-sphingosine (C24 Lactosyl(β) Ceramide, d18:1/24:0), and D-lactosyl-β1′-N-nervonoyl-D-erythro-sphingosine (C24:1 Lactosyl(β) Ceramide, d18:1/24:1).

In some embodiments, the sulfatide is selected from the group consisting of 3-O-sulfo-D-galactosyl-β1′-N-lignoceroyl-D-erythro-sphingosine (ammonium salt) (e.g., from porcine brain), 3-O-sulfo-D-galactosyl-β1′-N-lauroyl-D-erythro-sphingosine (ammonium salt) (C12 Mono-Sulfo Galactosyl(β) Ceramide, d18:1/12:0), 3-O-sulfo-D-galactosyl-β1′-N-heptadecanoyl-D-erythro-sphingosine (ammonium salt) (C17 Mono-Sulfo Galactosyl(β) Ceramide, d18:1/17:0), 3-O-sulfo-D-galactosyl-β1′-N-lignoceroyl-D-erythro-sphingosine (ammonium salt) (C24 Mono-Sulfo Galactosyl(β) Ceramide (d18:1/24:0), 3-O-sulfo-D-galactosyl-β1′-N-nervonoyl-D-erythro-sphingosine (ammonium salt) (C24:1 Mono-Sulfo Galactosyl(β) Ceramide, d18:1/24:1), and 3,6-di-O-sulfo-D-galactosyl-β1′-N-lauroyl-D-erythro-sphingosine (ammonium salt) (C12 Di-Sulfo Galactosyl(β) Ceramide, d18:1/12:0).

In some embodiments, the phosphospingolipid is selected from the group consisting of D-erythro-sphingosyl phosphoethanolamine (Sphingosyl PE, d18:1), N-lauroyl-D-erythro-sphingosyl phosphoethanolamine (C17 base) (C12 Sphingosyl PE, d17:1/12:0), and D-erythro-sphingosyl phosphoinositol (Sphingosyl PI).

In some embodiments, the phytosphingosine is selected from the group consisting of 4-hydroxysphinganine (Saccharomyces Cerevisiae) (D-ribo-Phytosphingosine), 4-hydroxysphinganine (C17 base) (D-ribo-phytosphingosine, C₁₇ base), 4-hydroxysphinganine-N,N-dimethyl (Saccharomyces Cerevisiae) (Phytosphingosine-N,N-Dimethyl), 4-hydroxysphinganine-N,N,N-trimethyl (methyl sulfate salt) (Saccharomyces cerevisiae) (Phyto sphingo sine-N,N,N-Trim ethyl), 4-hydroxy sphinganine-1-pho sphate (Saccharomyces Cerevisiae) (D-ribo-Phytosphingosine-1-Phosphate), 4-hydroxysphinganine-N,N-dimethyl- 1 -phosphate (ammonium salt) (Saccharomyces Cerevisiae) (Phytosphingosine-N,N-Dimethyl-1- Phosphate), N-acetoyl 4-hydroxysphinganine (Saccharomyces Cerevisiae) (N-02:0 Phytosphingosine), N-octanoyl 4-hydroxysphinganine (Saccharomyces Cerevisiae) (N-08:0 Phytosphingosine), N-stearoyl 4-hydroxysphinganine (Saccharomyces Cerevisiae) (N-18:0 Phytosphingosine), and 4-hydroxy sphinganine-1-phosphocholine (Saccharomyces Cerevisiae) (Phytosphingosine Phosphocholine).

Some aspects relate to a combination of a compound of Formula (I), (II), or (III) with one or more compounds selected a macrolide, a retinide, and a DES1 inhibitor. In some embodiments, the one or more retinide is fenretinide, N-(4-hydroxyphenyl) retinamide (4-HPR), 4-oxo-N-(4-hydroxyphenyl) retinamide (4-oxo-HPR), or motretinide. In some embodiments, the DES 1 inhibitor is selected from N-[(1R,2S)-2-hydroxy-1-hydroxymethyl-2-(2-tri decyl-1-cyclopropenyl)ethyl] octanamide (GT011) and (Z)-4-((5-(4-chlorophenyl)-1,3,4-oxadiazol-2-yl)amino)-N′-hydroxybenzimidamide (B-0027). In some embodiments, the one or more macrolide is selected from the group consisting of rapamycin, erythromycin, clarithromycin, roxithromycin, azithromycin, fidaxomicin, carbomycin A, josamycin, kitasamycin, midecamycin, oleandomycin, solithromycin, spiramycin, troleandomycin, tylosin, roxithromycin, telithromycin, cethromycin, solithromycin, solithromycin, tacrolimus, pimecrolimus, sirolimus, ciclosporin, polyene antimycotics, and cruentaren.

### Methods of Use

This disclosure provides for reversing hepatic steatosis comprising providing a consumable composition comprising at least one carrier. In accordance with such methods, an effective amount of an extract comprising a composition as described herein is provided to a subject in need thereof thereby reversing hepatic steatosis in a subject. The term “subject” as used herein refers to an animal, preferably a mammal. In some embodiments, the subject is a veterinary, companion, farm, laboratory or zoological animal. In other embodiments, the subject is a human.

In some aspects, administering a composition comprising a compound of Formula (I), Formula (II), or Formula (III), or a pharmaceutically acceptable salt thereof, isomer, homodimer, heterodimer, or conjugate, reverses hepatic steatosis. In some embodiments, a composition comprising a compound of Formula (I), Formula (II), or Formula (III) treats or ameliorates a disease or condition associated with reverses hepatic steatosis in a subject. In some embodiments, a composition comprising a compound of Formula (I), Formula (II), or Formula (III) treats or ameliorates a disease or condition associated with hepatic steatosis in a subject. In some embodiments, a composition comprising a compound of Formula (I), Formula (II), or Formula (III) treats or ameliorates a disease or condition associated with hepatic steatosis.

In an embodiment, administering a composition comprising a compound of Formula (I), Formula (II), or Formula (III), or a pharmaceutically acceptable salt thereof, treats or improves at least one factor associated with hepatic steatosis of a subject. In other aspects, a composition comprising a compound of Formula (I), Formula (II), or Formula (III), or a pharmaceutically acceptable salt thereof disclosed herein reverses hepatic steatosis of a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%, or ranges including and/or spanning the aforementioned values. In yet other aspects, a composition comprising Formula (I), Formula (II), or Formula (III), or a pharmaceutically acceptable salt thereof, improves hepatic steatosis a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

This disclosure further provides for promoting fat clearance comprising providing a consumable composition comprising at least one carrier. In accordance with such methods, an effective amount of an extract comprising a composition as described herein is provided to a subject in need thereof thereby promoting fat clearance in a subject. The term “subject” as used herein refers to an animal, preferably a mammal. In some embodiments, the subject is a veterinary, companion, farm, laboratory or zoological animal. In other embodiments, the subject is a human.

In some aspects, administering a composition comprising a compound of Formula (I), Formula (II), or Formula (III), or a pharmaceutically acceptable salt thereof, isomer, homodimer, heterodimer, or conjugate, promotes fat clearance. In some embodiments, administering a composition comprising a compound of Formula (I), Formula (II), or Formula (III) treats or ameliorates a disease or condition associated a fatty liver in a subject. In some embodiments, administering a composition comprising a compound of Formula (I), Formula (II), or Formula (III) treats or ameliorates a disease or condition associated with non-alcoholic fatty liver in a subject. In some embodiments, a composition comprising a compound of Formula (I), Formula (II), or Formula (III) treats or ameliorates a disease or condition associated with a fatty liver.

In an embodiment, a composition comprising a compound of Formula (I), Formula (II), or Formula (III), or a pharmaceutically acceptable salt thereof, treats or improves at least one factor associated with hepatic steatosis of a subject. In other aspects, a composition comprising a compound of Formula (I), Formula (II), or Formula (III), or a pharmaceutically acceptable salt thereof disclosed herein reverses hepatic steatosis of a subject by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects, a composition comprising Formula (I), Formula (II), or Formula (III), or a pharmaceutically acceptable salt thereof, improves hepatic steatosis a range from, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A subject in need of a composition of this disclosure includes a subject with observable symptoms associated with a fatty liver, as well as a subject who has no observable symptoms of a fatty liver but has been determined to be susceptible to developing a fatty liver. A subject in need of a composition of this disclosure includes a subject with observable symptoms associated with a non-alcoholic fatty liver, as well as a subject who has no observable symptoms of a fatty liver but has been determined to be susceptible to developing a non-alcoholic fatty liver.

The term “effective amount” as used herein means an amount of the compound, extract, or formulation containing the compound or extract, which is sufficient to significantly improve a disorder. As used herein, the term “improve” or “improved” should be taken broadly to encompass improvement in an identified characteristic of a disease state, said characteristic being regarded by one of skill in the art to generally correlate, or be indicative of, the disease in question, as compared to a control, or as compared to a known average quantity associated with the characteristic in question. For example, “improved” fat clearance associated with application of a compound or extract of the disclosure can be demonstrated by comparing the liver of a healthy subject with the liver of the liver of the subject in need. Alternatively, one could compare the fatty liver of a subject treated with a compound or extract of the disclosure to the average liver of a subject, as represented in scientific or medical publications known to those of skill in the art. In the present disclosure, “improved” does not necessarily demand that the data be statistically significant (i.e., p<0.05); rather, any quantifiable difference demonstrating that one value (e.g., the average treatment value) is different from another (e.g., the average control value) can rise to the level of “improved.”

Of concern when determining an effective amount to be used in humans is balancing the desired effects (benefits) against risks associated with use of a compound. At issue for such risk/benefit assessments is the types of adverse effects observed and the likelihood that they will occur.

In general, a suitable daily dose of a compound or extract of the disclosure will be that amount of a compound or extract which is the lowest dose that is effective at producing a desired benefit, in this case an effect that improves digestive health and consequently overall health and well-being. Such an effective dose will generally depend upon the factors described herein. For oral administration, the dose may range from about 0.0001 mg to about 10 grams per kilogram of body weight per day, about 5 mg to about 5 grams per kilogram of body weight per day, about 10 to about 2 grams per kilogram of body weight per day, or any other suitable dose. If desired, the effective daily dose of the compound or extract may be administered as two, three, four, five, six or more sub-doses administered separately at appropriate intervals throughout the day, optionally, in unit dosage forms. In some embodiments, dosing is one administration per day.

The compound or extract of the disclosure can be used alone or in combination with a particular diet or standard of care. By way of illustration, a compound or extract of this disclosure may be combined with a gluten-free diet, or used in combination with an aminosalicylate, a corticosteroid, athiopurine, methotrexate, a JAK inhibitor, a sphingosine 1-phosphate (SIP) receptor inhibitor, an anti-integrin biologic, an anti-IL12/23R or anti-IL23 biologic, and/or an anti-tumor necrosis factor agent or biologic.

### EXAMPLES

The following non-limiting examples are provided to further illustrate the present disclosure.

### Example 1

### Effects of N-Trans-Caffeoyltyramine on DIO Mice

Using an assay for human insulin promoter activity, which is highly sensitive to HNF4a activity, it was discovered that HMF4α activity is repressed by fatty acids. HNF4α is mutated in MODY1, an autosomal dominant monogenic form of diabetes, providing human genetic evidence for a direct role in diabetes pathogenesis. It is autoregulated through a positive feedback loop and is downregulated in T2D and NAFLD, as expected if lipotoxic effects of fatty acids repressed HNF4α activity.

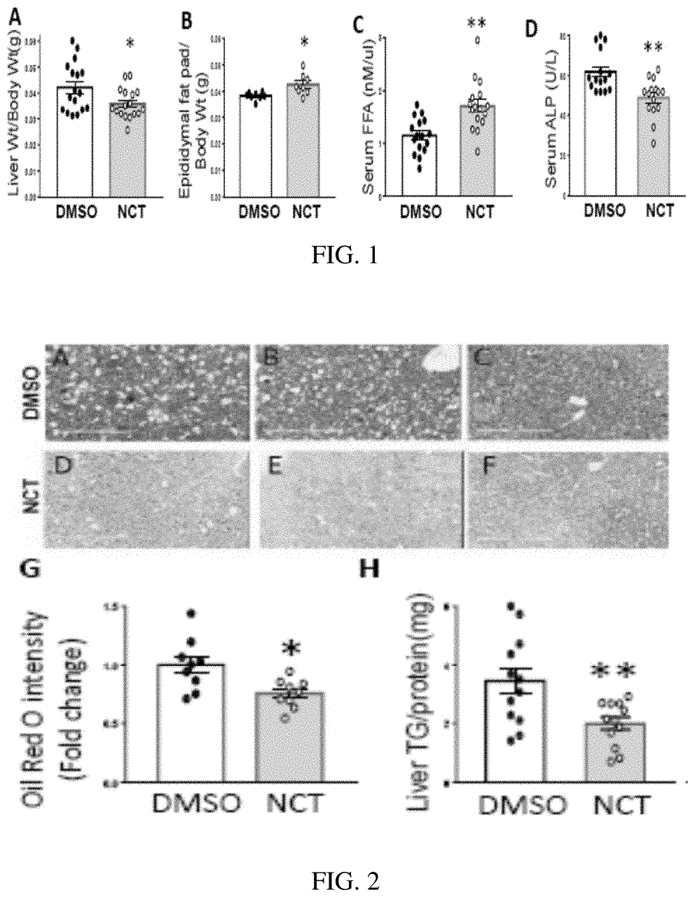

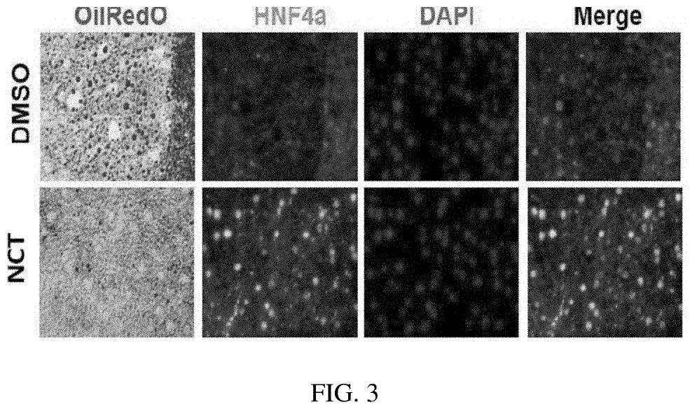

Compounds were injected IP into diet-induced obese mice at a dose of 200 mg/kg bid for two weeks. At that point, mice were sacrificed and organs were harvested for analysis. To test the hypothesis that HNF4a controls hepatic fat storage, N-trans-caffeoyltyramine was administered to C₅₇BL/6J DIO mice maintained on a 60% fat calorie diet. Based upon PK studies with N-trans-caffeoyltyramine, IP injection was chosen for two week for these proof of concept studies. After two weeks, the livers of the N-trans-caffeoyltyramine injected mice weighed less than those of the control mice. It was observed that N-trans-caffeoyltyramine was stimulating lipophagy (FIG. 1A). Further, there was a shift in the color of the liver from yellow to red. It was observed that with a decrease in fat content, and the hepatic triglyceride content was decreased (FIG. 2H). Analysis of liver sections revealed a decrease in stored fat by Oil Red O staining (FIG. 2, compare A-C with D-F, quantified in G). Both control and N-trans-caffeoyltyramine injected mice gained weight to a similar degree and both groups remained healthy and active.

The lack of a difference in body weight combined with the decreased liver weight implied that fat released from the liver must be present elsewhere. Consistent with that, there was an increase in epididymal fat pad mass (FIG. 1B). The shift of fat from the liver to adipose tissue implied that fatty acids had to have traveled through the circulation. This would be expected if lipophagy was being induced by N-trans-caffeoyltyramine, as lipophagy involves the release of fatty acids from cells through the action of LAL, which should lead to an increase in circulating free fatty acids (FFA). To test that prediction, a protocol was employed in which a glucose challenge is used to stimulate insulin secretion, which inhibits FFA release from adipocytes, leaving liver-derived FFA as the major source of circulating FFA. N-trans-caffeoyltyramine was induced an increase in free fatty acids (FIG. 1C).

Mechanistically, it was noted that N-trans-caffeoyltyramine induced the expression in T6PNE of genes involved in sphingolipid metabolism, particularly SPNS2, which has been studied most as a transporter of sphingosine-1 -phosphate (SIP). Knockdown of SPNS2 expression ablated fat clearance by N-trans-caffeoyltyramine, but S1P did not have any effect on fat storage. However, dihydroceramide was active at inducing fat clearance in the absence of N-trans-caffeoyltyramine. This required the expression of S1P receptors, indicating that dihydroceramide can act through the same receptor. Dihydroceramide was induced by N-trans-caffeoyltyramine through a pathway involving delta 4-desaturase, sphingolipid 1 (DES1).

The mechanism by which N-trans-caffeoyltyramine induced fat clearance was through induction of lipophagy, a type of autophagy that involves fusion of lipid vesicles with lysosomes, where the lipid vesicle triglycerides are degraded by lysosomal acid lipase. The lysosomal acid lipase inhibitor Lalistat 2 eliminated the ability of N-trans-caffeoyltyramine to effect fat clearance.

Previously, it was found that the HNF4a antagonist BI6015 caused loss of HNF4a expression in the liver and HNF4a is thought to play an important role in NAFLD. In control DIO mice, HNF4a protein was decreased, but N-trans-caffeoyltyramine reversed that reduction (FIG. 3). This is consistent with the in vitro finding that N-trans-caffeoyltyramine induces the expression of HNF4a mRNA.

N-trans-caffeoyltyramine promotes fat clearance from the steatotic livers of mice fed a high fat diet by inducing lipophagy, demonstrating a role for HMF4α in controlling the level of hepatic fat storage. On the basis of their potent in vivo effects and lack of toxicity, these new agonists appear to be strong candidates for NAFLD therapeutics. Further, subsequent studies have shown that the oral administration of N-trans-caffeoyltyramine is effective in reducing hepatic steatosis in mice.

### Example 2

### Liver Fat Storage and Potent HNF4α Agonist

### Materials and Methods

The T6PNE insulin promoter assay has been described previously and was performed here with slight modifications as follows: T6PNE cells were seeded at 200 cells per well in 384-well tissue culture plates (Greiner Bio-One) in the presence of 0.5 μM tamoxifen. Compounds described herein in DMSO were dispensed with an Echo 555 Acoustic Liquid Handler (Beckman Coulter). Three days after compound addition, cells were fixed in 4% paraformaldehyde (USBio) for 15 min and stained with DAPI (0.167 μg/ml, Invitrogen). Blue (DAPI) and green (GFP) channels were imaged using a Celigo imaging cytometer (Nexcelom Bioscience). The number of GFPpositive cells was normalized to the DAPI-positive cell number and fold change calculated relative to the DMSO control.

T6PNE cells were maintained in RPMI (5.5mM glucose, Corning) supplemented with 10% fetal bovine serum (FBS, Sigma-Aldrich) and 1% penicillin-streptomycin (pen-strep, Gibco). Cells were maintained in 5% CO₂ at 37° C. For the insulin promoter assay, 0.5 μM tamoxifen (Sigma-Aldrich) was added to T6PNE cell culture media. HepG2 or HeLa cells were cultured in DMEM (high glucose, Corning) supplemented with 10% FBS and 1% pen-strep and maintained at 5% CO₂, 37° C.

Oil red O and Nile Red staining were used to measure lipid accumulation. Oil Red O staining was done as known in the art. Briefly, fixed cells were incubated with Oil Red O solution (Poly Scientific) for 3 h, followed by photomicrography (Olympus, IX71). For Nile Red staining, dye (1: 500 in PBS from 1 mg/ml in ethanol stock, Sigma) was added for 30 mins and DAPI added for 10 mins at room temperature. Quantification of Nile Red staining was done with a Celigo imaging cytometer (Nexcelom Bioscience). More than 4000 cells were analyzed for each quantification. The number of Nile Redpositive cells was normalized to the DAPI-positive cell number for each well and fold change was calculated relative to a DMSO control well.

Slides containing frozen liver tissue sections from mice were air dried for 10-20 min followed by rehydration in distilled water. Sections were immersed in absolute propylene glycol (Cat #151957, MP Biomedicals, LLC, USA) for 2 min followed by 0.5% in oil red O solution (Cat #K043, Poly Scientific R&D, USA) for 2 h. Slides were then differentiated in 85% propylene glycol solution, washed with dH₂O for 2 h, and mounted using glycerin jelly mounting medium. All slides were scanned at a magnification of 20× using the Aperio Scanscope FL system (Aperio Technologies Inc., Vista, Calif., USA). The liver area stained with oil red O was measured using image J software as described, with some modifications. Oil red O-stained liver images were opened in Image J software. Using the Analyze>Set Scale command, the scale bar of the images was set to 200 um. RGB images were then converted into gray scale images using the Image>Type>RGB Stack command and were split into red, blue and green channels. Using the Image>Adjust>Threshold command, the threshold was manually set to highlight the oil red O-stained lipid droplets in the green channel. The same threshold was used for all the images in all treatment groups and the % oil red O-stained area was obtained using the Analyze→Measure tool command. Fold change was calculated by normalizing the values to images from mice fed normal chow.

Palmitate (150 mM) (Sigma-Aldrich) was prepared in 50% ethanol and precomplexed with 15% fatty acid-free BSA (Research Organics, Cleveland, Ohio, USA) in a 37 C water shaker. BSA-precomplexed palmitate was used as a 12 mM stock solution for all assays with a final concentration of 0.25 mM palmitate in cell culture medium.

siRNAs were purchased from Ambion. For transfections, 24 μL of each siRNA (1 μM stock) was mixed with 90 μL of a 1:100 dilution of Lipofectamine RNAi MAX (Invitrogen, Waltham, Mass., USA) in Opti-MEM. Transfection was done in 24 well plates (Thermo Fisher Scientific, Waltham, Mass., USA), by incubation with cells for 30 min. at room temperature. One day after transfection, cells were transferred to 96 well plates (2000 cells per well) and incubated at 37° C. for an additional day. Forty-eight hours after transfection, either palmitate-BSA complex or BSA control plus or minus compounds was added for 2 days. For validation of siRNAs, transfected cells were harvested for RNA purification and QPCR for each gene 2 days after transfection.

Quantitative PCR (Q-PCR) from in vitro cell culture experiments used total RNA purified using RNeasy Kits (Qiagen). For liver tissue samples, total RNA was isolated using Trizol (Invitrogen) and prepared for RNAseq (mouse liver DMSO and NCT, N=3). cDNA was amplified using 3 μg of total RNA using qScript cDNA SuperMix (Quanta BioSciences, Beverly, Mass., USA). QPCR analysis was performed using SYBR® Select Master Mix (Applied Biosystems) and ABI 7900HT (Applied Biosystems, Thermo Fisher Scientific). The Ct values of mRNA expression were then normalized to the 18 s rRNA values and are expressed as fold change over samples from mice fed normal chow for in vivo experiments or DMSO controls for cell culture experiments.

DARTS assays were conducted as described in the art. HepG2 cells were treated with DMSO, BI6015, NCT, NFT at a concentration of 40 or 80 μM for 16 hr. Total cell protein was extracted and measured by BCA protein assay (Thermo Scientific). Each sample was split into two aliquots for proteolysis without (−) or with (+) Subtilisin (Sigma-Aldrich). Forty mg of cell lysate was incubated with or without protease (40 ng/ml subtilisin) for 35 min at room temperature.

Whole-cell extracts were prepared by incubation in RIPA buffer (Invitrogen) containing protease inhibitors (Calbiochem, San Diego, Calif.). Protein (40 mg) was separated on 12% or 16% Tri-Glycine gels (Invitrogen) and transferred to Immobilon P membrane (0.2 μm pore, Millipore). After 1 h in phosphate-buffered saline-Tween (PBST) with 3% milk, membrane was incubated with antibodies to HNF4α (mouse, Novus, 1:1000), LC₃B (rabbit, Novus, 1:500), p62 (SQSTM1, mouse, Santa Cruz, 1:1000) or β-actin (mouse, Santa Cruz, 1:2000), followed by secondary antibody conjugated to horseradish peroxidase (1:5000, Jackson Immune). Signal was revealed by ECL (Thermo) and imaged with a ChemiDoc MP imager (Bio-Rad).

The bicinchoninic acid (BCA) protein assay kit assay (Thermo scientific) was used to measure protein amount for DART, Western blotting and TG assays. Absorbance at 550 nm was determined using a plate reader.

12-week-old C₅₇BL/6 J DIO male mice (cat #380050) were purchased from Jackson Laboratory and were fed with high fat diet containing 60 kcal % fat (Research Diets cat #D12492). Mice were maintained in a 12-h light/day cycle. After 2 weeks of acclimation, mice with similar body weights were randomly assigned to treatment or control groups.

For dose response experiments, mice were injected intraperitoneally with 10% DMSO as vehicle control or 4 different doses of NCT (30, 60, 120 and 240 mg/kg of body weight) dissolved in 10% DMSO. Mice received 2 doses per day with a 5 h interval between injections for 3 days. To test the effect of NCT (Sundia MediTech Company, Ltd., Custom synthesis), 200 mg/kg was injected IP bid for 14 days. On day 15, mice received a final dose of NCT followed by IP injection with 3 g/kg dextrose. One h later, blood samples were collected and mice were euthanized using pentobarbital. Total mouse, liver and epididymal fat pad weights were measured. Dissected liver samples were washed in cold PBS, cut into small pieces and distributed for analyses. For RNA isolation and ELISA, liver samples were snap frozen using liquid nitrogen and stored at −80° C. For histomorphometry and immunofluorescence analysis, liver samples were fixed in 4% of cold PFA and processed for histology. All animal experiments were approved by the Institutional Animal Care and Use Committee (IACUC) of the Sanford Burnham Prebys Medical Discovery Institute in accordance with national regulations.

Frozen liver sections were permeabilized using 0.3% Triton-X and incubated in antigen retrieval solution (Antigen retrieval citrate, Biogenex) at sub boiling temperature for 10 min. Subsequently, sections were incubated with blocking buffer containing 5% normal donkey serum (Jackson Immuno Research) followed by incubation overnight at 4° C. with mouse monoclonal primary antibody against HNF4 (1:800, Cat #PP-H1415-00, R&D Systems). Sections were washed and incubated with anti-mouse secondary antibody coupled with Alexa flour 488 (1:400, Invitrogen) or with DyLight 647 (1:400, Jackson Immuno) for 1 h at room temperature and counterstained with DAPI (40,6-diamidino-2-phenylindole, Sigma Aldrich). For lipid droplet staining, slides were incubated in Bodipy 500/510 (1:100 from 1 mg/ml, 4,4-Difluoro-5-Methyl-4-Bora-3a,4a-Diaza-s-Indacene-3-Dodecanoic Acid, Invitrogen) for 30 min. Slides were mounted using fluorescence mounting medium and images were obtained at 40× magnification using an Olympus IX71 fluorescence microscope. Fluorescence intensity of HNF4α-stained nuclei was calculated using MetaMorph TL software (version 7.6.5.0, Olympus).

The serum FFA level was measured using the Free Fatty Acid Quantification Colorimetric/Fluorometric Kit (Cat #K612, BioVision, USA). Fold change was calculated by normalizing to values from mice fed normal chow.

Serum and liver TG level was measured using the Triglyceride calorimetric Assay Kit (Cat #10010303, Cayman Chemicals, USA). Fold change was calculated by normalizing the values from normal chow mice.

100 μL of whole blood was collected in lithium heparin blood collection tubes and transferred to single use VetScan mammalian liver profile reagent rotors. The levels of multiple analytes present in the blood samples were quantified using a VetScan VS2 Chemistry Analyzer (Abaxis North America, USA).

The alkaline phosphatase level in serum samples was quantified using a Catalyst One Chemistry Analyzer (IDEXX Laboratories, Inc. USA).

Microsomal stability studies were performed in the Conrad Prebys Center for Chemical Genomics. In vitro metabolism was conducted in a system consisted of NADPH generating system, test compound, and Tris·Cl buffer. The mixture was pre-incubated at 37° C. for 30 min. Reactions were initiated by addition of mouse or human liver microsomes suspension and shaken at 37° C. with air exposure. To generate the stability curve for the test compound, the incubation was terminated at 0, 5, 15, 30 and 60 min. NFT and NCT concentrations were determined by LC-MS. The result of metabolic stability was expressed as the percentage of compound remaining at 1 hr. The in vitro half-life (t1/2) and intrinsic clearance (Clint) were calculated based on drug depletion over incubation time.

Murine PK was conducted by WuXi AppTec (Shanghai, China). C₅₇BL/6 male mice of age 7-9 weeks were obtained from SLAC Laboratory Animal Co (Shanghai, China). Mice were fasted for 12 h prior to compound administration. Oral gavage was used for PO dosing. For IV dosing, compound was administered by tail vein injection. For compound concentration determination, 25 μL of blood was collected from the submandibular or saphenous vein and processed for plasma. Plasma concentration of compound was determined by LC-MS/MS.

Ceramides and dihydroceramides were measured in the UCSD Lipidomics Core Facility as previously described (Quehenberger et al.). Briefly, samples were extracted using the butanol: methanol (BUME) method. The lipid layer was collected and run on a Thermo-Vanquish UPLC (Thermo Scientific) with a Cortecs T3 (C₁₈), 2.1 mm×150 mm; 1.8 uT3 column and a binary solvent system. Mass spectrometry employed a Thermo Q Exactive instrument with MS/MS data dependent acquisition scan mode and LipidSearch software (Thermo Fisher Scientific).

Lipid nomenclature is given for the validated species: Cer d32:1_19.14 d18:1/n14:0. d stands for dihydroxy and stands for tri-hydroxy; d32:1 indicates that the sum total carbons is 32 and the species contains 1 double bond; the number following the underscore is the retention time; d18:1/n14:0 indicates that indicates that the sphingoid base fatty acid is 18:1 and contains 2 hydroxy groups; 14:0 is the amide bonded fatty acid that contains no (n) hydroxy group; n14:0 indicates that 14:0 is the amide bonded fatty acid that contains no (n) hydroxy group.

STRING (https://string-db.org) shows protein-protein interaction networks. The top 50 gene candidates upregulated in NCT treated mouse liver (N=3) were analyzed. STRING functional enrichment analysis was also performed.

Experiments with primary human hepatocytes were performed by CN-Bio (Cambridge, UK). Primary human hepatocytes (PHHs), human Kupffer cells (HKs) and human stellate cells (HSCs) were seeded onto CN-Bio's PhysioMimix LC₁₂ MPS culture plates at 6×10⁵ cells for PHHs and 6×10⁴ cells for HKs and HSCs in 1.6 ml of CN-Bio's HEP-lean media with 5% FCS. Throughout the experiment the cells were maintained at a flow rate of 1 μl/s. After 24 h (Day 1) of seeding, the media was changed to HEP-lean media and the cells were incubated until day 4 to allow the cells to form microtissues. At day 4 post seeding, media was changed to HEP-fat media and treated with DMSO or NCT (5, 15, 40 μM). Media was replaced on days 6 and 8. Cells were harvested on day 10 for RNA extraction. Data are presented as mean ±SEM of three or more samples as indicated. Statistical significance was assessed using Student's t-test, ANOVA or R² coefficient of correlation.

### Results and Discussion

Using the insulin promoter assay, it was found that fatty acids inhibit HNF4α activity, a previously undescribed activity, even though it was known that fatty acids bind in the HNF4α ligand binding pocket. Also using the insulin promoter assay, it was discovered both antagonists and agonists of HMF4α. The HNF4α activators were alverine and benfluorex, known drugs that have been used for irritable bowel syndrome and weight loss/type 2 diabetes, respectively. Of note, benfluorex has been studied in clinical trials for type 2 diabetes and proved to be effective at reducing HbA1c. Unfortunately, both alverine and benfluorex were relatively weak activators, making it difficult to study the role of HNF4α in lipotoxic diseases.

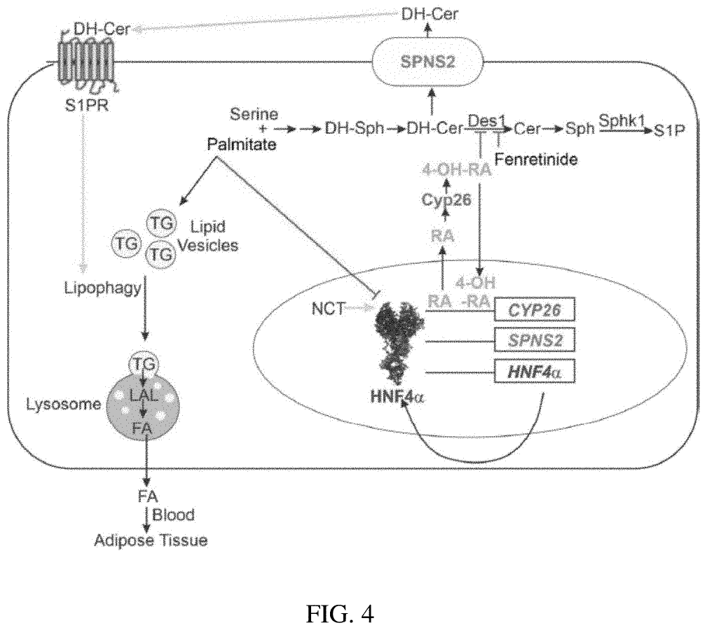

To find more potent HNF4α activators, the compounds were examined that had structural similarity with alverine or benfluorex. The plant-based compounds N-trans caffeoyltyramine (NCT), and N-trans feruloyltyramine (NFT) which are associated with plant cell walls as part of a damage response, were found to be more potent activators of the human insulin promoter-GFP transgene in T6PNE cells. Of note, NFT is derived from NCT by the action of caffeoyltyramine-O-methyltransferase. NCT and NFT induced clearance of fat from the T6PNE cells used in the insulin promoter assay and NCT was studied in vivo, where it reversed hepatic steatosis. The mechanism by which HNF4α affects hepatic fat storage is induction of lipophagy, a form of autophagy that involves fusion of lipid droplets with lysosomes and lipid hydrolysis through lysosomal acid lipase. Not wanting to be bound by theory, this is a mechanism distinct from that regulating adipocyte fat storage through hormone-sensitive lipase. The data presented here demonstrate that HNF4α meets both of the principal requirements for a molecule that can mediate the control of hepatic lipid storage. It senses fat directly by fatty acid binding to the HNF4α ligand binding pocket (LBP), which controls HNF4α activity. The level of HNF4α activity then determines the extent of lipophagy, which releases fat from lipid vesicles in hepatocytes, thus regulating the amount of fat stored in the liver (FIG. 4).

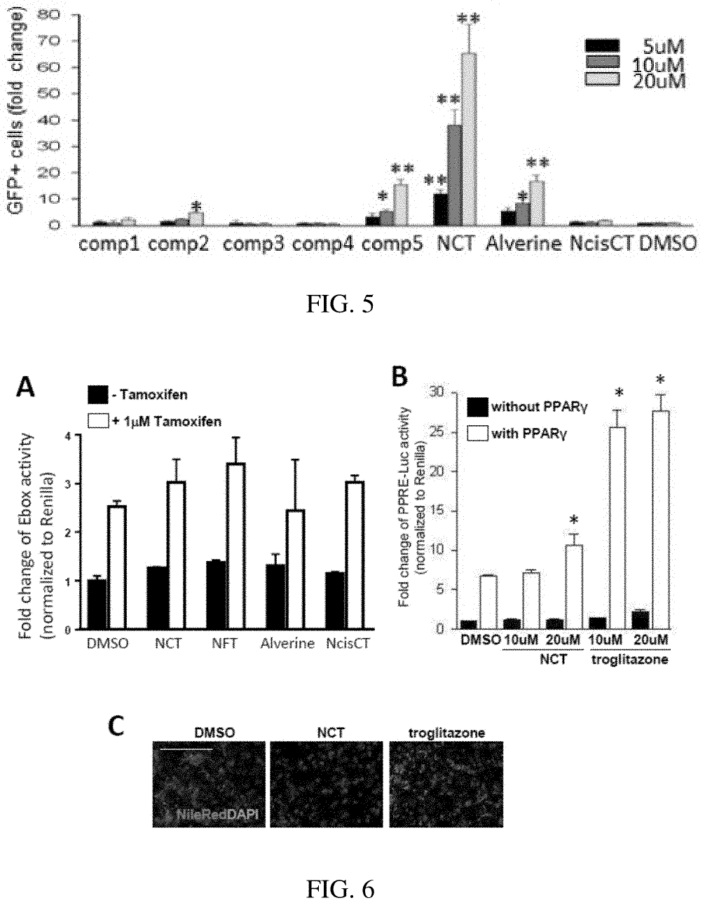

A library was screened of known drugs and discovered that alverine and benfluorex, which are structurally similar but used for completely distinct indications, were activators of HNF4α. Because alverine and benfluorex were fairly weak HNF4α activators, finding stronger activators was compelling. Thus, some compounds were tested that had structural similarity with alverine and benfluorex to find additional HNF4α activators. N-trans caffeoyltyramine (NCT) and N-transferuloyltyramine (NFT) were reproducibly positive hits. NCT was the strongest inducer of insulin promoter activity, while NFT was approximately as active as alverine. As expected for nuclear receptor ligands, which are well-known for having highly sensitive structure-activity relationships, small structural differences resulted in large changes in activity (FIG. 5). NCT, which was more potent than NFT, differs from NFT by a single methyl group. In plants, NCT is converted to NFT by caffeoyltyramine-O-methyltransferase, resulting in generally higher levels of NFT than NCT. There was no estrogenic or PPARγ receptor agonist activity, both of which can produce false positives in the assay (FIG. 6).

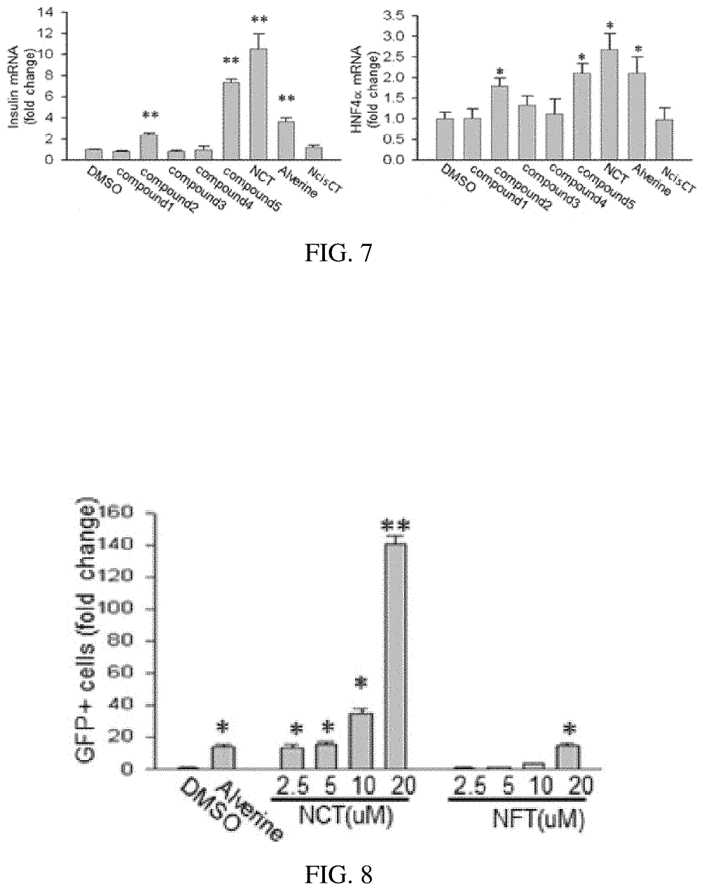

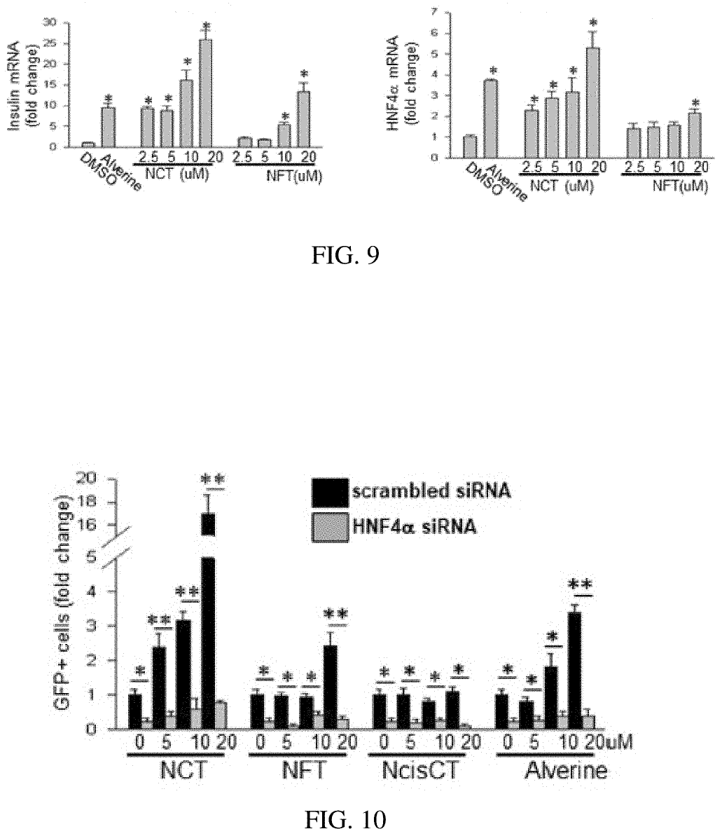

Both NCT and NFT, and to a lesser degree N-p-coumaroyltyramine, increased INS and HNF4α mRNA (FIG. 7). The increase in HNF4α mRNA is particularly important, since HNF4α gene expression is the best indicator of HNF4α activity in our hands, as the protein acts on its own promoter through a positive feedback loop. Both NCT and NFT exhibited dose responsiveness in the INS promoter assay (FIG. 8) and with respect to their ability to increase INS and HNF4α mRNA levels (FIG. 9). It is likely that fatty acids and the HNF4α agonists compete for occupancy of the HNF4a LBP and that this could account for threshold effects seen in the dose-response curves.

### NCT and NFT Act Directly on HNF4α

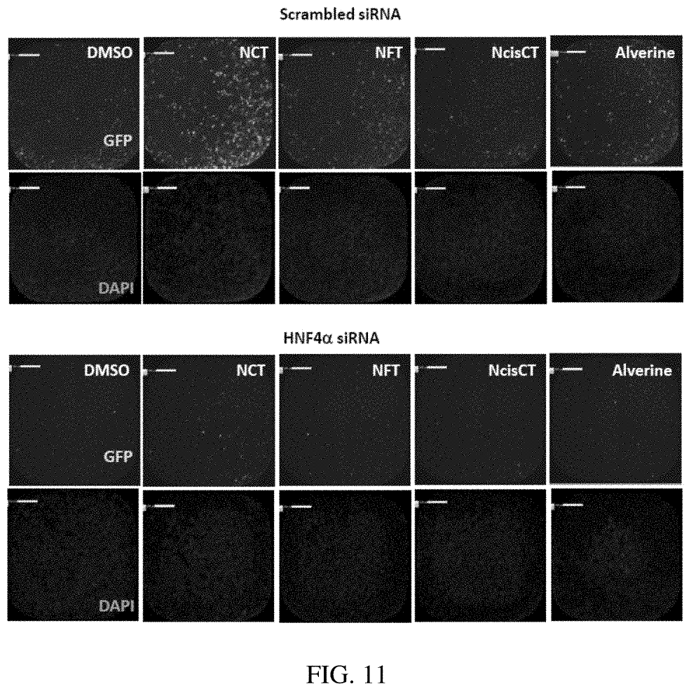

A prediction if the compounds act on HNF4α is that HNF4α siRNA should ablate their effect. Consistent with that prediction, HNF4α siRNA repressed the effect of NCT and NFT on the INS promoter (FIG. 10 and FIG. 11).

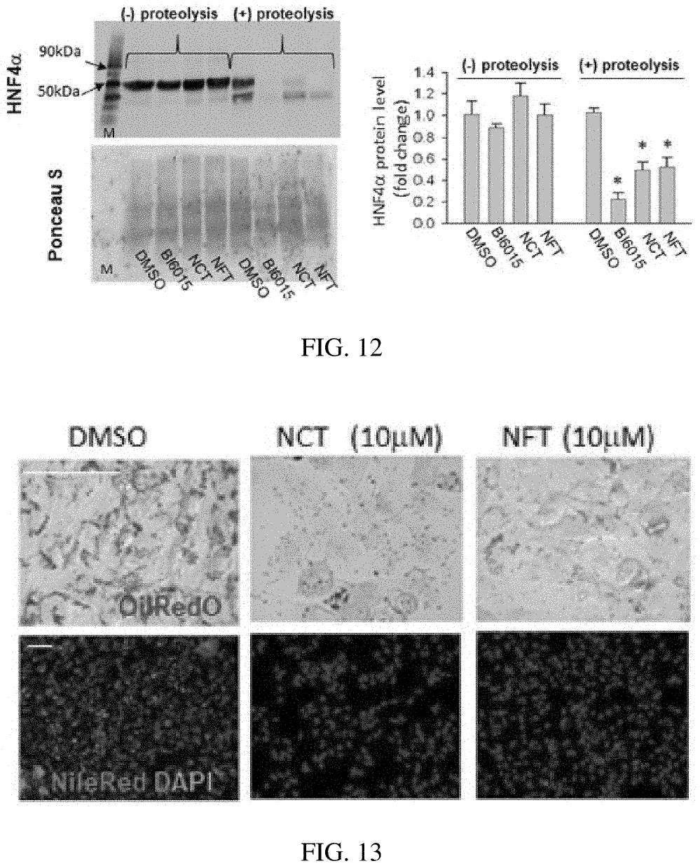

Binding of a compound to its target is expected to alter the structure of the target protein. This can often be detected as a change in the sensitivity to proteolytic cleavage, which is the basis for the DARTS assay. It was previously determined whether compounds induce a conformational change in HNF4α, thus demonstrating a direct effect on the protein. Consistent with our previous results, the potent HNF4α antagonist BI6015 induced a conformational change in HNF4α (FIG. 12). NCT and NFT also induced a change in HNF4α proteolytic sensitivity, as expected if they act directly (FIG. 12).

NCT Induces Fat Clearance from Cells

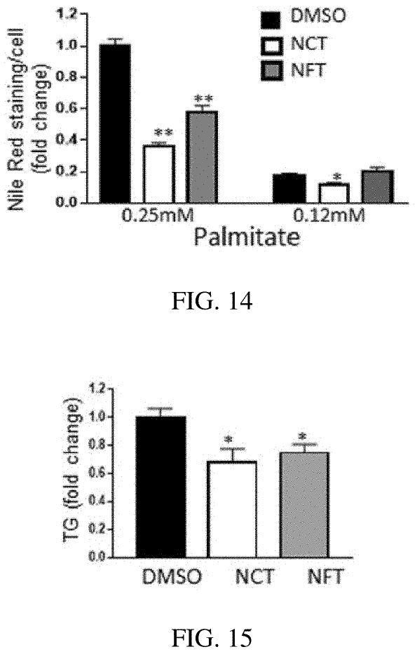

The HNF4α antagonist BI6015 caused hepatic steatosis in vitro and in vivo, and genetic deletion of HNF4α leads to hepatic steatosis25. Alverine and benfluorex were ascertained in a modification of the insulin promoter assay in which the level of insulin promoter activity was repressed with palmitate. In that modified assay, alverine and benfluorex reversed the fatty acid-mediated repression of the human insulin promoter. Thus, it was logical to ask whether more potent HNF4α agonists could alleviate steatosis. Cells were treated with 0.25 mM palmitate for 2 days in the presence and absence of NCT or NFT (10 μM). As demonstrated by Oil Red O and Nile Red staining, cells treated with NCT or NFT had less stored fat than control cells (FIG. 13, quantified in FIG. 14, FIG. 6). This was further demonstrated by quantification of the cellular triglyceride (TG) level (FIG. 15), which produced the same result as the Nile Red staining, validating the accuracy of that assay.

### The S1P Transporter SPNS2 is Required for Fat Clearance by NCT

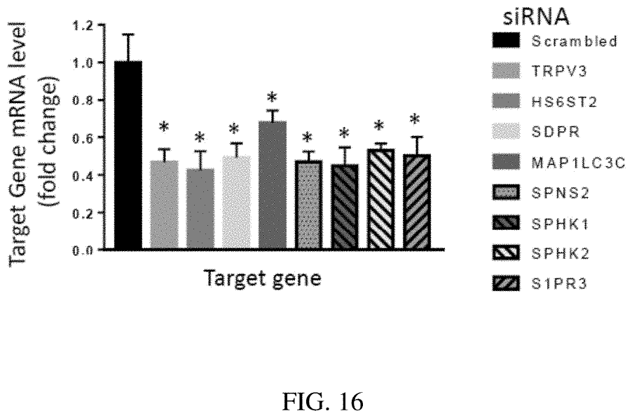

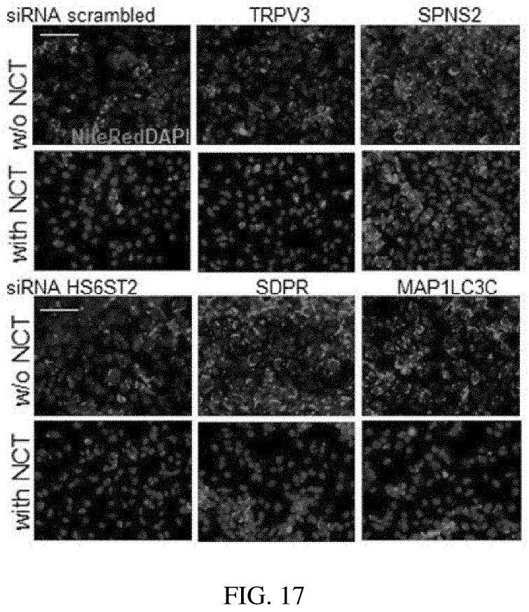

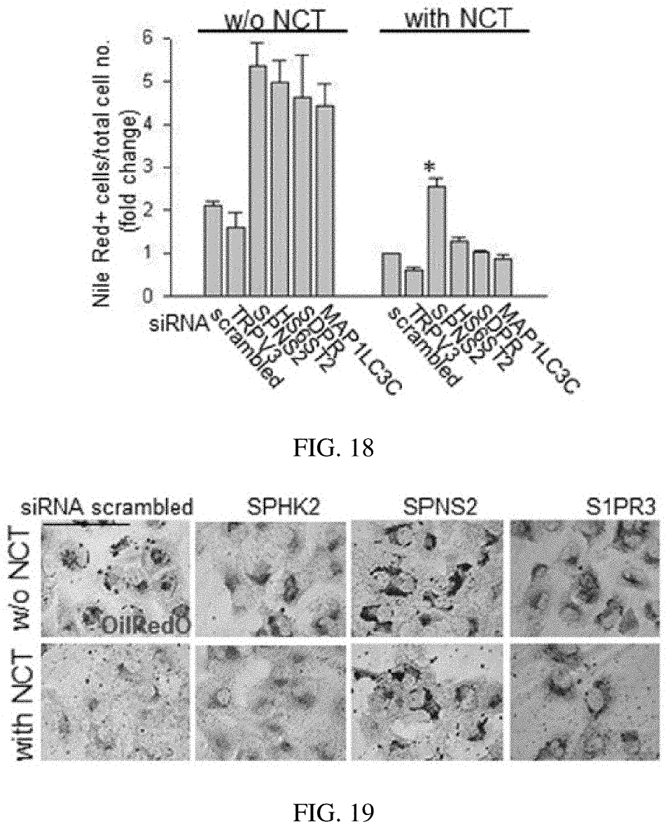

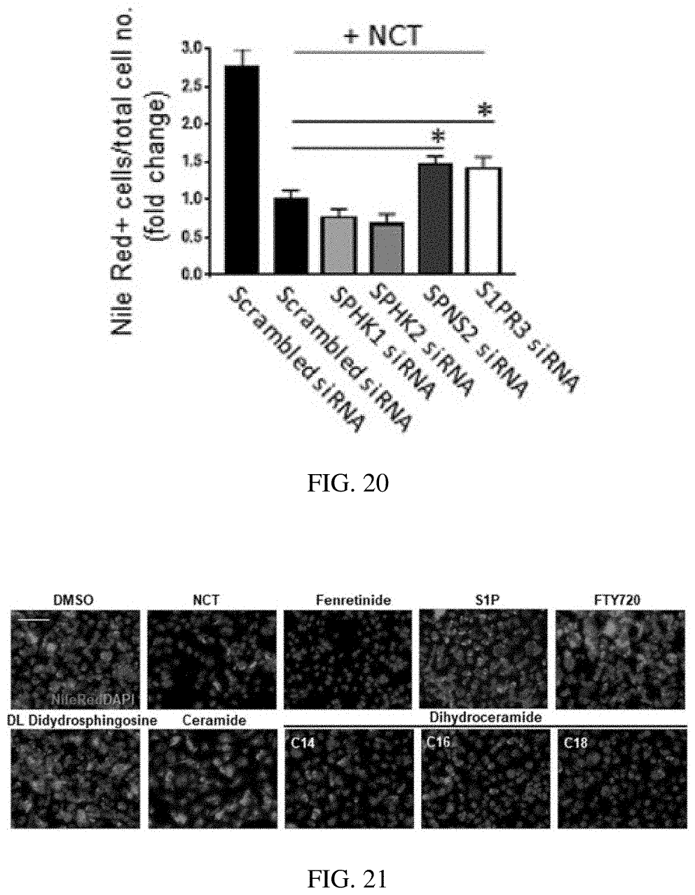

Given that HNF4α is a transcription factor, the mechanism by which the newly discovered HNF4α agonists caused reversal of cellular steatosis would involve genes downstream of HNF4α that are regulated by HNF4α. Genes with mRNA levels that were affected by both NCT and NFT and that were involved in lipid metabolism were tested for a role in fat clearance downstream of HNF4α by inhibiting their expression using siRNA (siRNA validation in FIG. 16). Of the genes tested, siRNA to only one, SPNS2, blocked the effect of NCT on fat clearance (FIG. 17, quantified in FIG. 18). SPNS2 encodes a transporter for sphingosine-1-phosphate (SIP) that moves it from the intracellular to the extracellular space. Of note, palmitate, which was used to induce steatosis and which is the major fat consumed by humans is a precursor for S1P synthesis. An S1P analog that causes immunosuppression, (FTY720, aka fingolimod), has been approved for the treatment of multiple sclerosis. S11³ is synthesized from sphingosine by the action of sphingosine kinases (Sphk1,2). T6PNE cells express only Sphk2 to a significant degree (GEO Accession GSE18821, GSE33432) and siRNA to Sphk2 had no effect on fat clearance induced by NCT (FIG. 19, quantified in FIG. 20) eliminating S1P as the molecule responsible for inducing fat clearance.

Once transported outside cells by SPNS2, S1P binds to a receptor in the GPCR family of signaling receptors, of which there are five family members (S1PR1-5). S1PR signaling plays an important role in diverse cell processes, but particularly in immune responses. Only one member of the S1PR family, S1PR3, is expressed in T6PNE cells (GEO Accession GSE18821, GSE33432). S1PR3 siRNA blocked the ability of NCT to induce fat clearance (FIG. 19, quantified in FIG. 20), consistent with a model in which a molecule transported by SPNS2 that then stimulates S1PR signaling triggers fat clearance.

### Dihydroceramides are Required for Fat Clearance by NCT

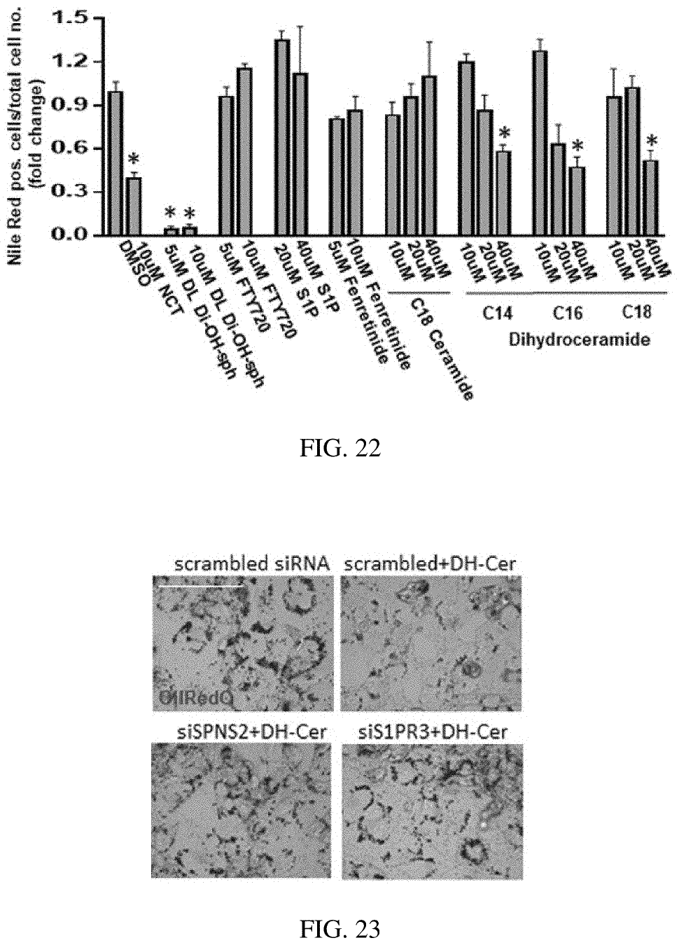

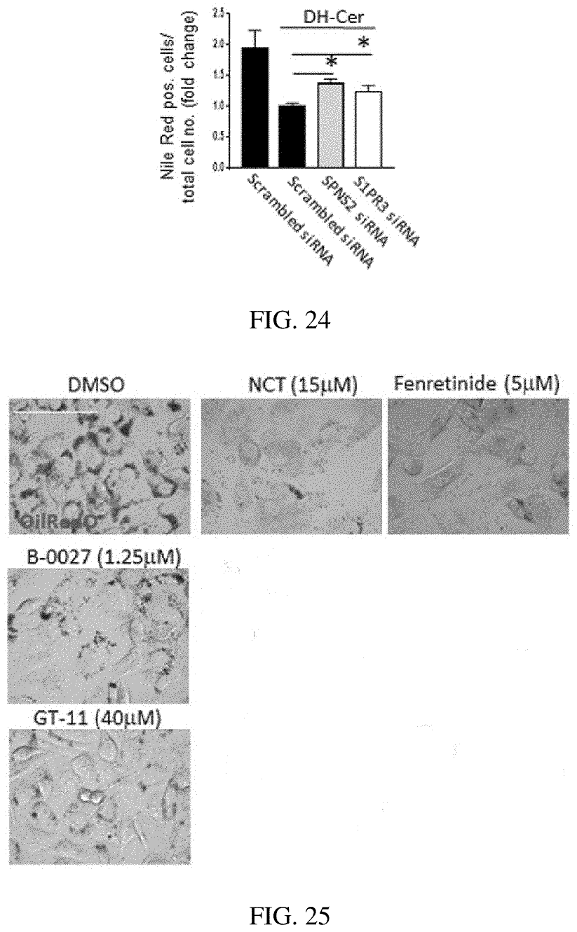

To our surprise, neither S1P nor FTY720 had any effect on fat clearance (FIG. 21, quantified in FIG. 22). However, because of the known roles of SPNS2 and S1PR3, molecules structurally related to S1P and that could be acted on by SPNS2 and S1PR3 were the obvious candidates for being the effectors in fat clearance induced by NCT. De novo S1P biosynthesis begins with palmitate and serine and proceeds through dihydrosphingosine, dihydroceramide, ceramide, and sphingosine (FIG. 4). Dihydrosphingosines have been shown to be transported by SPNS2, but had no effect on fat clearance (FIG. 21, quantified in FIG. 22). In contrast, multiple dihydroceramides were highly effective at inducing fat clearance (FIG. 21, quantified in FIG. 22). This suggests that, like S1P and dihydrosphingosines, dihydroceramides are transported by SPNS2 and act through S1PRs to effect fat clearance from cells (FIG. 23, quantified in FIG. 24).

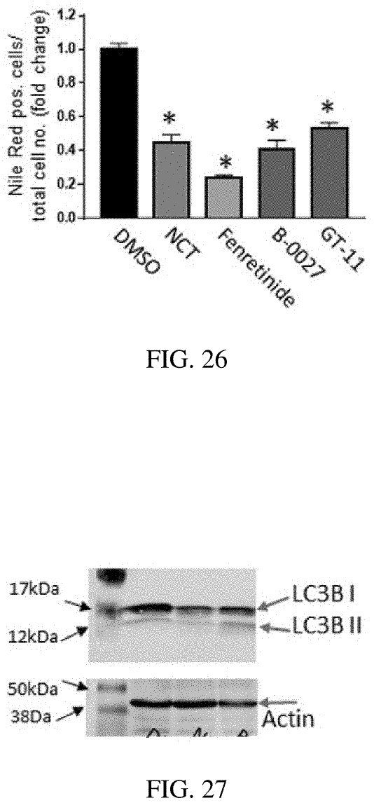

If dihydroceramides are the active molecule in fat clearance, inhibition of their conversion to ceramides by dihydroceramide desaturase 1 (DES1) should increases their level and promote fat clearance (FIG. 4). Fenretinide is a synthetic retinoid derivative that inhibits DES1 but it has multiple other targets as well. It was strongly positive in the fat clearance assay (FIG. 21, quantified in FIG. 22), as were the more specific DES1 inhibitors GT-11 and B-0027 (FIG. 25, quantified in FIG. 26) This provides further evidence that dihydroceramides are the active species responsible for the ability of NCT to induce fat clearance from cells.

### NCT Promotes Fat Clearance by Inducing Lipophagy

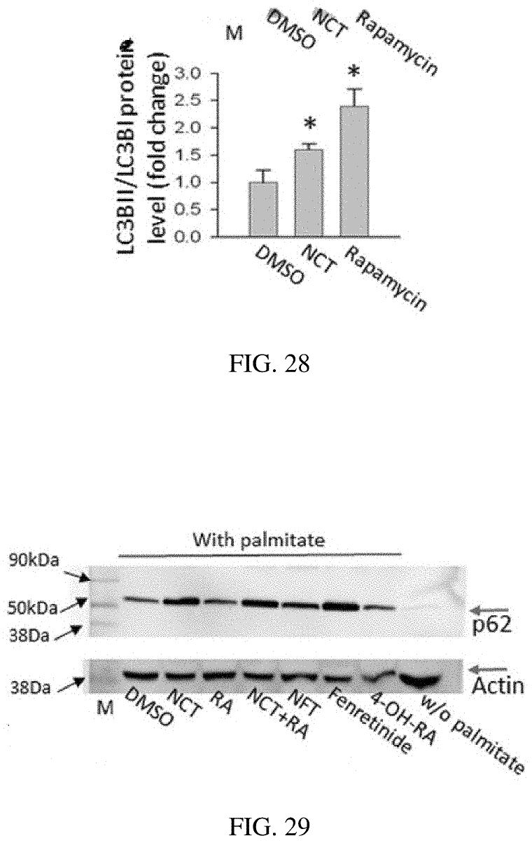

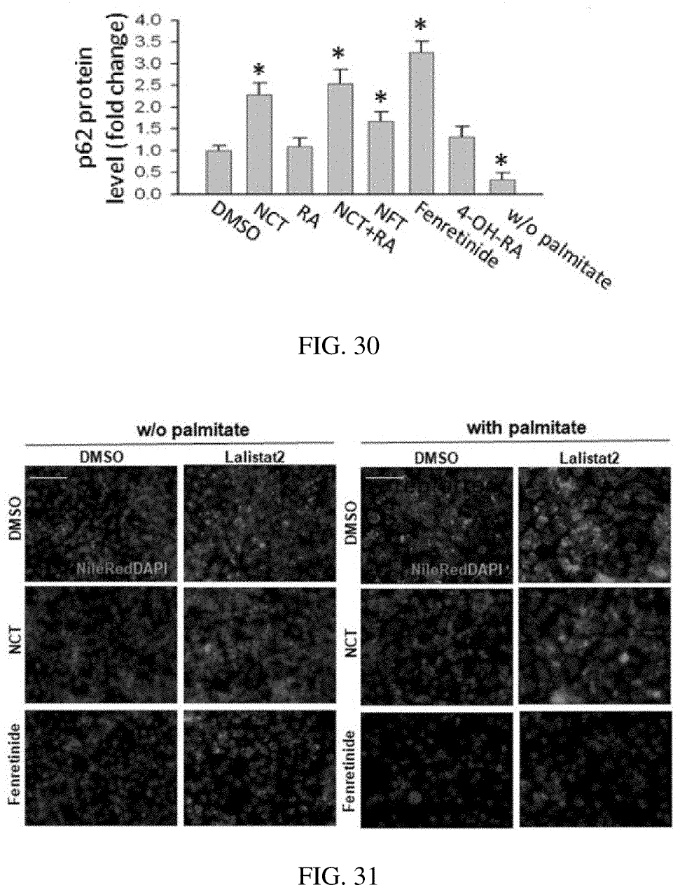

The ability of dihydroceramides to effect fat clearance from cells raised the question of the mechanism by which they act. Some studies have found a role for dihydroceramides in autophagy. Autophagy involves alterations in LC3B through lipidation and effects on the level of the p62 autophagy receptor that can be monitored by Western blotting, with the polarity of those changes being complex and varying under different circumstances and in different cells. Contrary to the classical situation in which p62 varies inversely with autophagic flux, but similar to rapamycin, which was used as a positive control to induce autophagy, NCT induced increases in the LC₃B-II to LC₃B-I ratio (FIG. 27, quantified in FIG. 28), and in p62 (FIG. 29, quantified in FIG. 30).

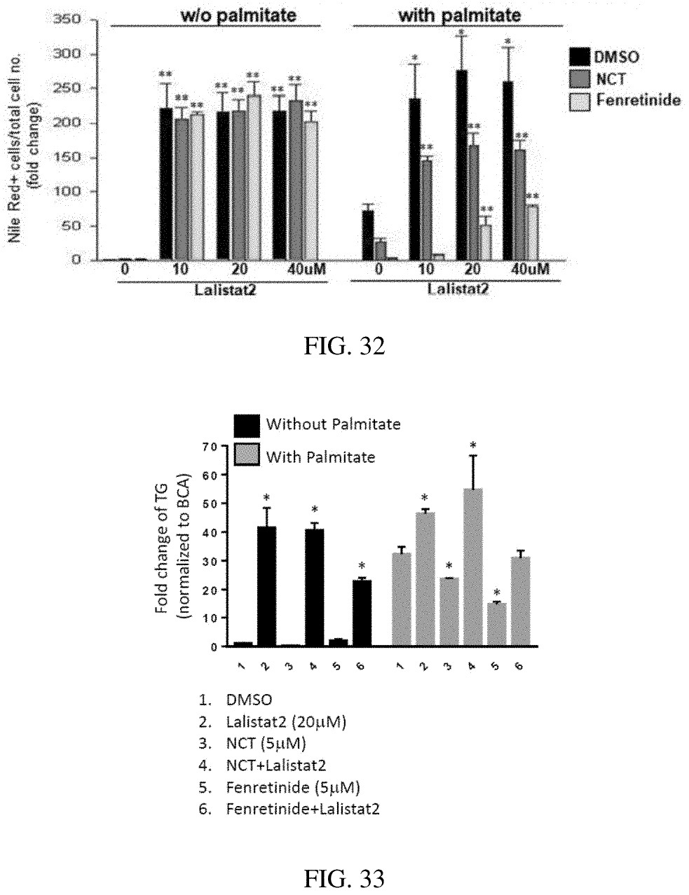

Lipophagy is a form of autophagy that has been associated with dihydroceramides. A central aspect of lipophagy is the cleavage of triglycerides from lipid droplets by lysosomal acid lipase (LAL). Direct association between lipid droplets and lysosomes has been demonstrated. To determine whether lipophagy plays a role in fat clearance induced by NCT, the LAL inhibitor Lalistat 2 was used. Consistent with NCT acting by stimulation of lipophagy, Lalistat 2 inhibited the ability of NCT to promote fat clearance (FIG. 31, quantified in FIG. 32, FIG. 33).

### NCT Reverses Hepatic Steatosis

Having established that NCT reduces the level of stored fat in cells in vitro, it was of interest to determine its effect in vivo. The liver was focused on as the organ expressing the highest level of HNF4α and a major site of pathological fat storage, i.e., NAFLD. HNF4α has been recognized as playing an important role in NAFLD. Adipose cells, the other major site of fat storage, do not express HNF4α. NCT was administered by IP injection (200 mg/kg bid) for two weeks to C₅₇BL/6 J DIO mice maintained on a 60% fat calorie diet. The dose was selected on the basis of a dose response study in which mice were injected with NCT at increasing doses (30, 60, 120 and 240 mg/kg bid for 3 days), which was well tolerated.

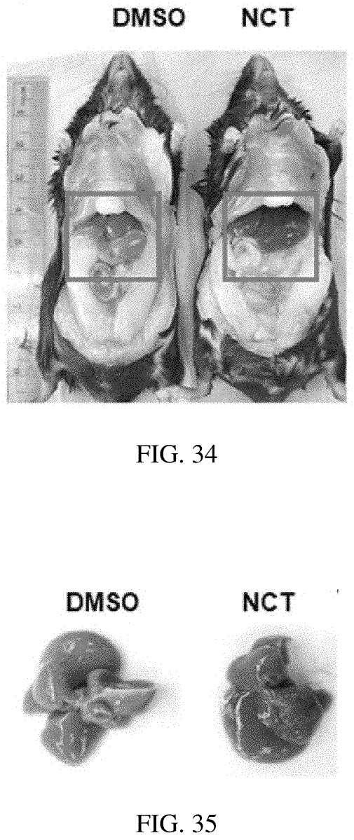

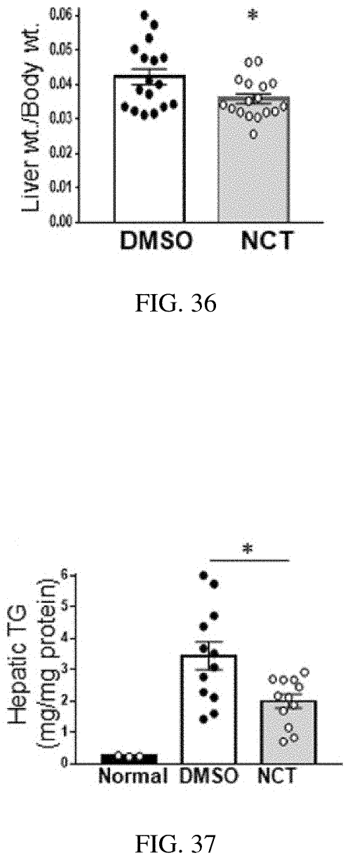

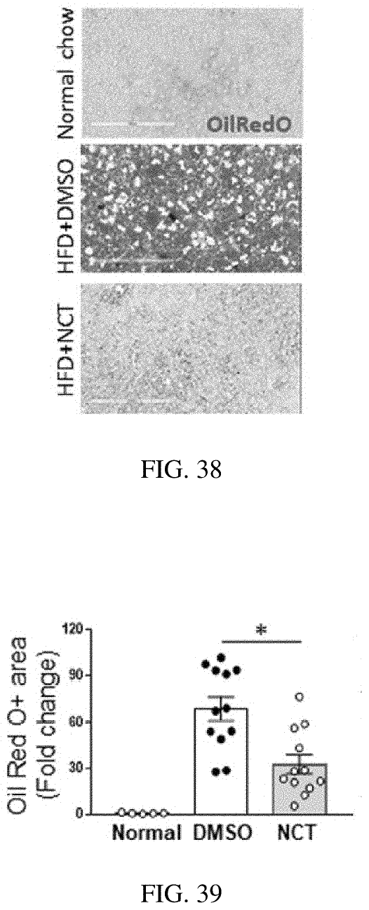

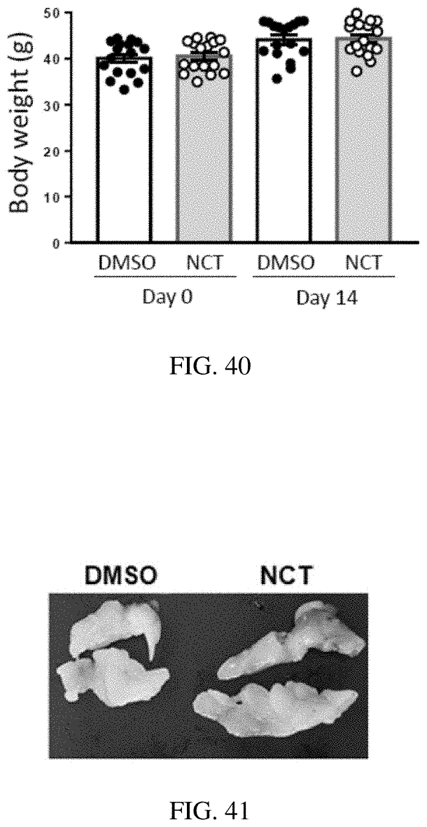

After two weeks, the livers of the NCT-injected mice exhibited a shift in the color of the liver from yellow to red, as expected with a decrease in fat content (FIG. 34 and FIG. 35). Livers from NCT-injected mice weighed less than those of the control mice, as expected if NCT was stimulating loss of hepatic fat (FIG. 36) and this was borne out by a reduction in the hepatic triglyceride content (FIG. 37). Analysis of liver sections revealed a decrease in stored fat by Oil Red O staining (FIG. 38 and FIG. 39). Control and NCT-injected mice gained weight to a similar degree and both groups remained healthy and active (FIG. 40).

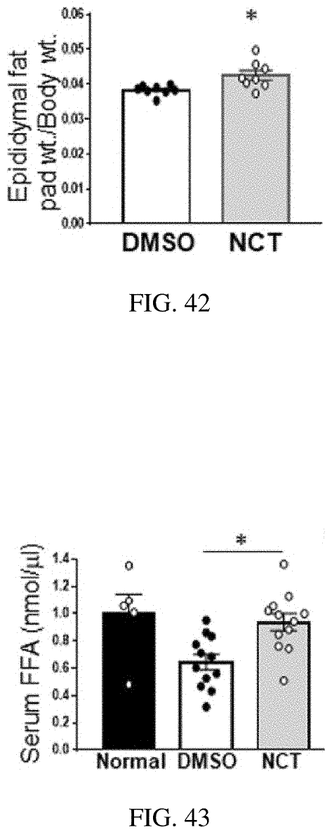

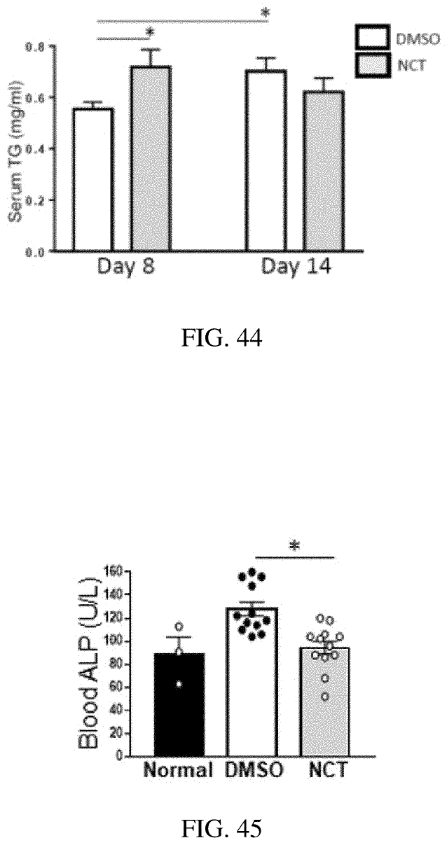

The lack of a difference in body weight combined with the decreased liver weight implied that fat released from the liver must be present elsewhere. Consistent with that, there was an increase in epididymal fat pad mass (FIG. 41 and FIG. 42). The shift of fat from the liver to adipose tissue suggested that fatty acids had to have traveled through the circulation to shift from one tissue to another. This would be expected if lipophagy was being induced by NCT, as lipophagy involves the release of fatty acids from cells through the action of LAL, which should lead to an increase in circulating free fatty acids (FFA). To test that prediction, a protocol was employed that was used in humans and mice in which a glucose challenge is used to stimulate insulin secretion, which inhibits FFA release from adipocytes, leaving liver-derived FFA as the major source of circulating FFA. Consistent with our hypothesis, NCT induced an increase in free fatty acids (FIG. 43). Serum TG was increased by NCT at week 1 of NCT administration but not at week 2, the end of the study (FIG. 44).

### ALP was Decreased by NCT

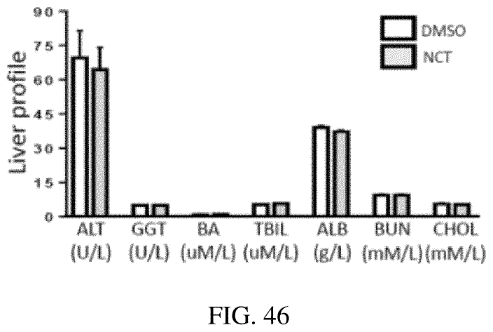

Markers of liver injury are elevated in NAFLD, including alkaline phosphatase (ALP) ALP has been studied as an early indicator of the transition to hepatic fibrosis as part of the progression from NAFLD to non-alcoholic steatotic hepatitis (NASH). Mice treated with NCT exhibited decreased ALP (FIG. 45). There was no change in the level of other markers in the VetScan panel (FIG. 46).

### NCT Reverses Negative Effects of Fatty Acids on HNF4α Expression

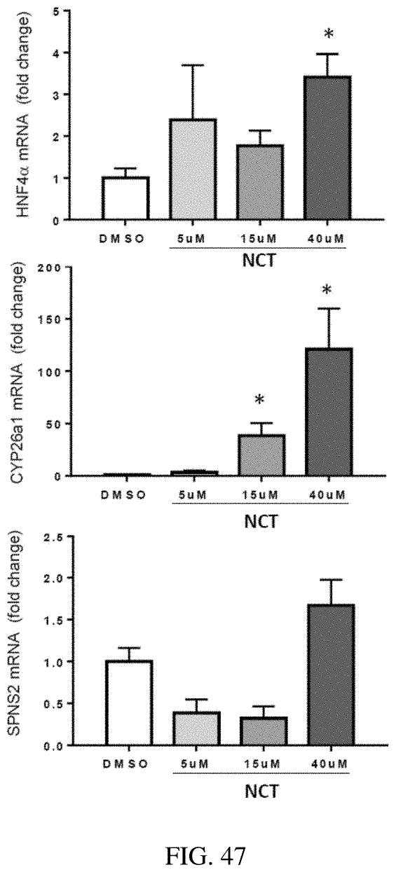

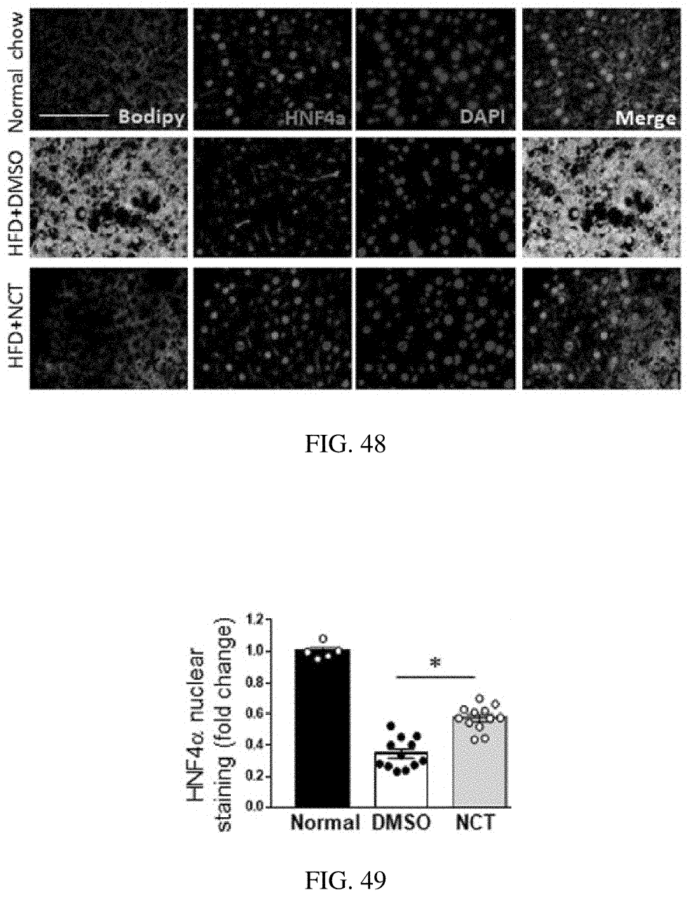

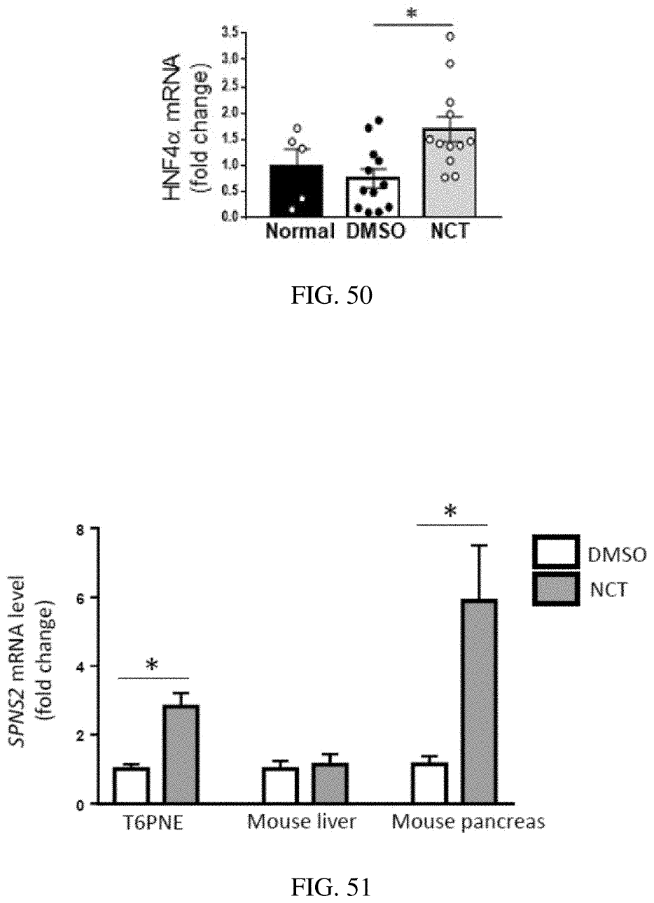

HNF4α feeds back on its own promoter in a positive feedback loop and is the best marker of HNF4α activity in our experience. In vitro, NCT and NFT induced HNF4α expression in T6PNE cells (FIG. 9) and primary human hepatocytes (FIG. 47). In vivo, it was shown that the potent HNF4α antagonist BI6015 caused loss of HNF4α expression in the liver, so it was tested whether NCT had an effect in vivo. In control DIO mice, HNF4α protein was decreased compared with mice on a normal chow diet, as expected given the previous finding that fatty acids inhibit HNF4α activity (FIG. 48 and FIG. 49). Interestingly, it was not detected that a decrease in HNF4α mRNA with HFD (FIG. 50), which could be due to post-translational regulation of HNF4α. NCT reversed the loss of HNF4α protein (FIG. 48 and FIG. 49) and mRNA (FIG. 50).

### CYP26A1 Plays an Important Role in the Induction of Fat Clearance

In the mouse pancreas and T6PNE cells, NCT induced SPNS2 expression (FIG. 51), which is required for the reversal of cellular steatosis (see, FIGS. 13-18). However, NCT did not induce a change in SPNS2 expression in the mouse liver (FIG. 51) or primary cultured human hepatocytes (FIG. 47). Because it was deemed unlikely that NCT was acting by a fundamentally different mechanism to clear fat from the liver than in T6PNE cells in vitro which was originated from human pancreas, it was examined for other genes induced by NCT in the liver that might be involved in the same pathway. CYP26A1 is induced by HMF4α in conjunction with retinoic acid (RA) and converts RA to multiple metabolites, including 4-oxo-RA, 5,6 epoxy-RA, and 4-OH-RA. RA is synthesized in the liver and so is abundant there.

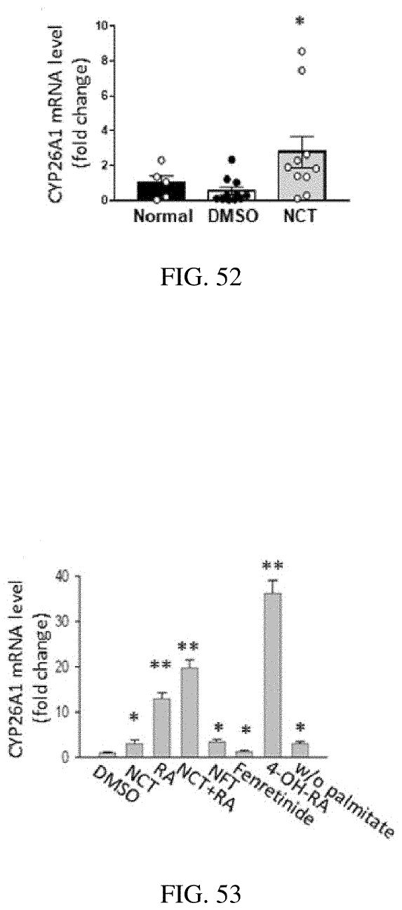

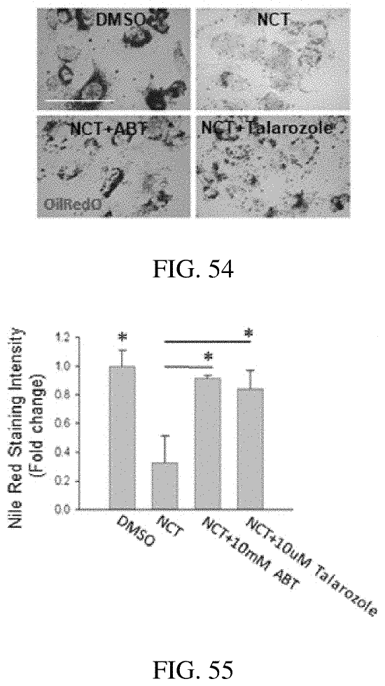

It was shown that fenretinide, a synthetic retinoid that inhibits DES1, induced fat clearance from T6PNE cells, it was hypothesized that CYP26A1 was a good candidate for playing a role in NCT-induced fat clearance from the liver. NCT induced CYP26a1 in the mouse liver (FIG. 52), cultured human hepatocytes (FIG. 47) and in T6PNE cells (FIG. 53). The generalized CYP inhibitor ABT and the specific CYP26 inhibitor talarazole inhibited fat clearance by NCT (FIG. 54 and FIG. 55).

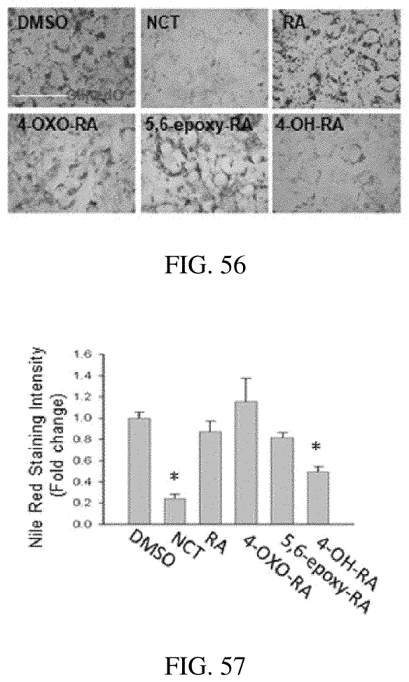

Having demonstrated a role for CYP26A1 in fat clearance downstream of NCT, it was hypothesized that one of the retinoic acid metabolites produced by CYP26 should induce fat clearance. Consistent with that, 4-OH RA induced fat clearance from T6PNE cells (FIG. 56 and FIG. 57). Interestingly, 4-OH-RA also induced an increase in CYP26A1 mRNA (FIG. 53) Thus, RA and its metabolites play important roles in fat storage in the liver through transcriptional and post-transcriptional mechanisms, respectively.

### NCT Induces Dihydroceramide Production

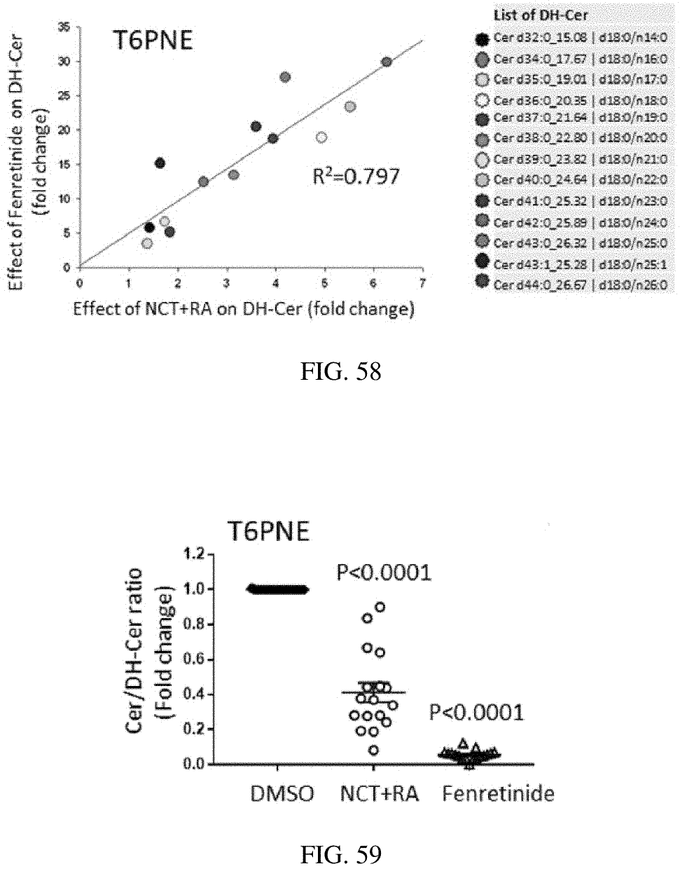

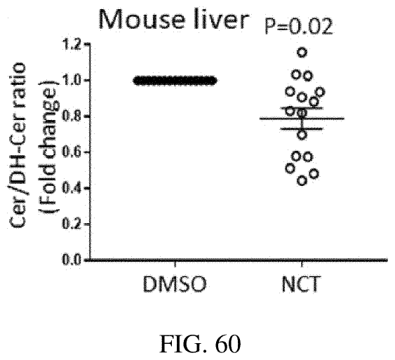

A key prediction of our model of the control of hepatic fat storage by HNF4α is that HMF4α should increase the production of dihydroceramides. This was tested in vitro and in vivo using a lipidomic approach. Lipidomic analysis revealed that multiple dihydroceramides were increased by NCT in T6PNE cells. Strikingly, there was a strong correlation between the dihydroceramides produced in response to NCT and those induced by fenretinide (FIG. 58, R²=0.79), consistent with the model (FIG. 4) that a downstream effect of NCT is to inhibit DES1. Both NCT+RA (used to induce CYP26A) and fenretinide induced a substantial decrease in the ratio of ceramide produced by the action of DES1 relative to the corresponding dihydroceramide (FIG. 59). This was evident as well in lipidomic analysis of livers from mice injected IP with NCT (FIG. 60). This is consistent with NCT administration in vivo resulting in inhibition of DES1 and consequent increased dihydroceramide production in the liver as predicted by the model (FIG. 4).

Thus, the data provided above describes the discovery of a previously unknown pathway by which HNF4α controls hepatic fat storage. That discovery was made possible by the discovery of potent HNF4α agonists, also reported here. Stimulation of HNF4α activity with the HNF4α agonist NCT led to reversal of hepatic steatosis in the short time frame of two weeks. Encouragingly for its therapeutic potential, NCT treatment was completely nontoxic and led to a decrease in ALP, an important marker of liver injury and progression from NAFLD to NASH. ALP has been put forth as a marker of hepatic fibrosis and is also used commonly as a biomarker for biliary cholangitis.

The HMF4α agonists described here are structurally similar to alverine and benfluorex, known drugs that were found to be HNF4α activators. The strategy of drug repurposing that is being used for many diseases, including COVID-19, but there are few examples of success. Alverine and benfluorex are weak HNF4α activators and are unsuitable for in vivo use, but they served as an important starting point for the efforts described here, and thus can be taken as partial validation of the strategy. However, NCT and NFT are much more potent, making possible in vivo and mechanistic studies. NCT and NFT are found in plants, including some consumed by humans. They have no known role as nuclear receptor ligands or mediators of plant metabolism, and there is no HNF4α homolog in plants. There have been many reports of compounds found in plants and plant extracts that have beneficial effects on disorders caused by fat excess, including type 2 diabetes and fatty liver disease. NCT and NFT are found in association with plant cell walls. They are induced in response to damage and are thought to play a role in pathogen defense. However, little is known about their function. In animal cells and in mice, they have been shown to have anti-inflammatory properties. The concentration of NCT in most plants is low, as it is an NFT precursor, being converted to NFT by O-methylation of the 3-hydroxyl group of the phenylpropenic acid moiety. NFT is more abundant, being found in various plants at a few tens of micrograms per gm of dry plant, but that will vary depending on the degree to which the compounds were induced prior to harvest. Given their poor oral bioavailability and low abundance in plants that are commonly consumed as food, NCT and NFT are unlikely to be physiologically relevant sources of HNF4α ligands in most human diets, but that is a question worthy of additional investigation.

The mechanism that was found for the control of hepatic fat storage by HMF4α involves the induction of lipophagy, a form of autophagy. Genetic knockout studies and molecules that stimulate autophagy have implicated autophagy in fatty liver disease. However, the induction of lipophagy by HNF4α has not been suspected previously. An advantage of stimulating lipophagy by HNF4α agonists rather than by general stimulators of autophagy is that HMF4α expression is highly tissue restricted, being expressed predominantly in the liver, pancreas, kidney and intestine. It was not observed that any systemic effects of NCT, and in fact were unable to establish a maximum tolerated dose because of the lack of toxicity. The fat released from the liver appeared to be taken up by adipocytes, which do not express HNF4α.

HNF4α is a nuclear receptor transcription factor, so it was hypothesized that the mechanism by which it stimulates lipophagy must involve HNF4α-mediated transcriptional effects on genes that ultimately promote lipophagy. In the T6PNE cells used in the insulin promoter assay, upregulation by NCT of SPNS2, a transporter, led to the identification of dihydroceramides as playing a key role in stimulating lipophagy downstream of HMF4α. In the liver in vivo, HNF4α acts in conjunction with retinoic acid to activate CYP26A1 transcription. In one of the many feedback loops in this system, RA is then metabolized by CYP26 itself to a number of metabolites, of which it was found at least one, 4-OH RA, to inhibit the dihydroceramide metabolizing enzyme DES1 to increase the production of dihydroceramides. 4-OH RA also induces CYP26A expression. Complex and interacting feedback loops appear to be a central feature of the pathway described here. One of the most important of those is the inhibition by palmitate of HNF4α activity, which should lead to decreased lipophagy and consequent increased fat storage. However, de novo dihydroceramide synthesis begins with palmitate, the major fat consumed by humans and a major effector of lipotoxicity. It is showed here that dihydroceramides stimulate lipophagy, resulting in decreased fat storage, opposing its negative effect on HMF4α as a direct inhibitor through binding to HNF4α.

Dihydroceramides have been implicated in lipotoxic diseases, including hepatic steatosis and type 2 diabetes. They have been measured in the blood type 2 diabetes and cardiovascular disease. Genetic ablation of DES1 improves insulin resistance and hepatic steatosis, but dihydroceramide function in those diseases has not been well understood. Under our model, the highest concentration of secreted dihydroceramides will be in the immediate vicinity of their site of export by SPNS2, so it is likely that they act primarily in an autocrine and/or paracrine manner through S1PRs on nearby cells. It was found that S1PR3 is necessary for the action of NCT and dihydroceramides in T6PNE cells and so must be a receptor for dihydroceramides, but the other four receptors have not been studied in this regard. They are expressed in complex patterns, which could potentially contribute to effects in other tissues. For example, dihydroceramides play important roles in hematopoietic stem cells, so restricting activity primarily to the organs that are affected by a particular disease such as NAFLD by targeting HMF4α could potentially avoid undesirable side effects. An interesting area for future studies will be to determine how SIP receptors signal to lipid vesicles to promote lipophagy.

The model described above does not require the existence of an endogenous HNF4α agonist, and no such agonist has been found. Rather, HNF4α appears to exhibit a high level of basal activity in the absence of ligand binding, with fatty acids acting as endogenous antagonists. The high potency of NCT as an HNF4α agonist allows for activation of lipophagy even in the face of a high, pathological level of endogenous fat. The finding that fatty acids regulate HNF4α activity combined with the finding that HNF4α regulates lipophagy support a model in which HNF4α is repressed by high levels of ingested fat under fed conditions. This leads to suppression of hepatic lipophagy and storage of any available fat. Under the condition of starvation, HNF4α will not have bound fat, as found for linoleic acid, and thus will be more active, leading to increased hepatic lipophagy and release of stored fat. Ingestion of a high level of dietary fat will lead to constitutive downregulation of HNF4α activity and thus to hepatic steatosis.

In addition to hepatic steatosis, HNF4α is implicated in a number of other diseases, that affect tissues with high HNF4α expression, including type 2 diabetes, where HNF4α has been found to be a type 2 diabetes gene in a number of GWAS studies. Haploinsufficiency for HNF4α causes MODY1, a monogenic form of diabetes.

Thus, HNF4α agonists could be of benefit in other settings in addition to NAFLD. Of note, hepatic steatosis and consequent hepatic insulin resistance play an important role in T2D pathogenesis, so ameliorating NAFLD could have beneficial effects on diabetes independent of any effects on the islet. In the intestine, HNF4α has been implicated in inflammatory bowel disease by GWAS studies. A site of significant HNF4α expression is the kidney, and obesity is a strong risk factor for the development of renal disease. Thus, pharmacologic activation of HNF4α may be useful in diseases affecting organs other than the liver. The HNF4α activators studied here, NCT and NFT, differ in their respective abilities to activate the insulin promoter.

The disclosure is generally described herein using affirmative language to describe the numerous embodiments. The disclosure also includes embodiments in which subject matter is excluded, in full or in part, such as substances or materials, method steps and conditions, protocols, or procedures. Various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for reversing hepatic steatosis in a subject in need, the method comprising:
administering to the subject in need thereof an oral composition comprising at least one carrier and an effective amount of a compound of Formula (I), or an isomer, salt, homodimer, heterodimer, or conjugate thereof: 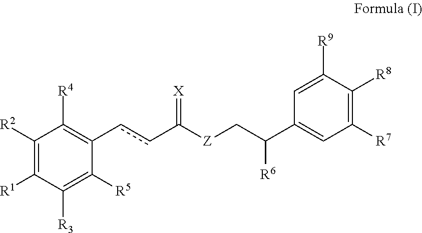
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionallysubstituted —(O)C₁₋₆ alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂-heterocyclyl, optionally substituted —(O)C₁₋₆alkylC₄₋₁₂-heterocyclyl, optionally substituted —(O)C₄₋₁₂aryl, optionally substituted —(O)C₁₋₆alkylC₅₋₁₂-aryl, optionally substituted —(O)C₁₋₁₂heteroaryl, and optionally substituted —(O)C₁₋₆alkylC₁₋₁₂heteroaryl,
thereby reversing hepatic steatosis.

2. The method of claim 1, wherein said compound has the structure of Formula II: 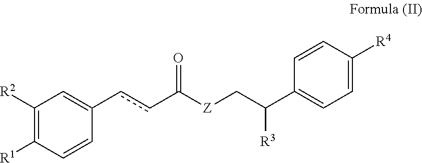
wherein:
R¹, R², R³, and R⁴ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkyl C₄₋₁₂ cycloalkyl, optionally substituted —(O)C₄₋₁₂heterocyclyl, optionally substituted —(O)C₁₋₆alkylC₄₋₁₂-heterocyclyl, optionally substituted —(O)C₄₋₁₂aryl, optionally substituted —(O)C₁₋₆alkylC₅₋₁₂-aryl, optionally substituted —(O)C₁₋₁₂-heteroaryl, and optionally substituted —(O)C₁₋₆alkylC₁₋₁₂-heteroaryl;
the dashed bond is present or absent;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂-heterocyclyl, optionally substituted —(O)C₁₋₆alkylC₄₋₁₂-heterocyclyl, optionally substituted —(O)C₄₋₁₂aryl, optionally substituted —(O)C₁₋₆alkylC₅₋₁₂-aryl, optionally substituted —(O)C₁₋₁₂heteroaryl, and optionally substituted —(O)C₁₋₆alkylC₁₋₁₂heteroaryl.

3. The method of claim 1, wherein the composition is formulated as a dietary supplement, food ingredient or additive, a medical food, nutraceutical or pharmaceutical composition.

4. The method of claim 1, wherein R¹, R², R³, and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂-heterocyclyl, optionally substituted —(O)C₄₋₁₂aryl, optionally substituted —(O)C₁₋₆alkylC₅₋₁₂ aryl, optionally substituted —(O)C₁₋₁₂heteroaryl, and optionally substituted —(O)C₁₋₆alkylC₁₋₁₂heteroaryl;
R⁴, R⁵, R⁶, R⁷, and R⁹ are each independently hydrogen, deuterium, hydroxyl, or halogen;
dashed bond is present;
X is O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₂₋₁₂heteroaryl, and optionally substituted —(O)C₁₋₆alkylC₁₋₁₂heteroaryl.

5. The method of claim 1, wherein R¹, R², and R⁸ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl;
R³, R⁴, R⁵, R⁶, R⁷, and R⁹ are each independently hydrogen, deuterium, hydroxyl, or halogen;
the dashed bond is present;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

6. The method of claim 2, wherein R⁴ is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂-heterocyclyl, optionally substituted —(O)C₁₋₆ alkyl C₄₋₁₂-heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl;
R¹ and R² are —OH;
R³ is H;
the dashed bond is present;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

7. The method of claim 2, wherein R² and R⁴ is selected from hydrogen, deuterium, hydroxyl, halogen, cyano, nitro, optionally substituted amino, optionally substituted C-amido, optionally substituted N-amido, optionally substituted ester, optionally substituted — (O)C₁₋₆alkyl, optionally substituted —(O)C₁₋₆alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkyl C₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl;
R¹ is —OH;
R³ is H;
the dashed bond is present;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂-heterocyclyl, optionally substituted —(O)C₁₋₆alkylC₄₋₁₂-heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

8. The method of claim 2, R¹, R², and R⁴ are —OH;
R³ is H;
the dashed bond is present;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂-heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

9. The method of claim 1, wherein the compound of Formula (I) or Formula (II) is selected from the group consisting of: N-trans-caffeoyltyramine, N-cis-caffeoyltyramine, N-trans-feruloyltyramine, N-cis-feruloyltyramine, p-coumaroyltyramine, cinnamoyltyramine, sinapoyltyramine, and 5-hydroxyferuloyltyramine, or a pharmaceutically acceptable salt, solvates, and combinations of the foregoing.

10. The method of claim 1, wherein the compound of Formula (I) or Formula (II) is selected from the group consisting of: a compound of Formula (II) is selected from (E)-3-(3,4-dihydroxyphenyl)-N-(4-ethoxyphenethyl)acrylamide, (E)-3-(3,4-dihydroxyphenyl)-N-(4-(2-methoxyethoxy)phenethyl)acrylamide, (E)-3-(3,4-dihydroxyphenyl)-N-(4-(2-(methylsulfonyl)ethoxy)phenethyl)acrylamide, (E)-2-(4-(2-(3-(3,4-dihydroxyphenyl)acrylamido)ethyl)phenoxy)acetic acid, ethyl (E)-2-(4-(2-(3-(3,4-dihydroxyphenyl)acrylamido)ethyl)phenoxy)acetate, (E)-N-(4-(cyclopropylmethoxy)phenethyl)-3-(3,4-dihydroxyphenyl)acrylamide, (E)-3-(3,4-dihydroxyphenyl)-N-(4-(3,3,3-trifluoropropoxy)phenethyl)acrylamide, (E)-3-(3,4-dihydroxyphenyl)-N-(4-((tetrahydro-2H-pyran-4-yl)methoxy)phenethyl)acrylamide, (E)-3-(3,4-dihydroxyphenyl)-N-(4-((4-fluorobenzyl)oxy)phenethyl)acrylamide, (E)-N-(4-(cyanomethoxy)phenethyl)-3-(3,4-dihydroxyphenyl)acrylamide, (E)-3-(3,4-dihydroxyphenyl)-N-(4-(pyridin-3-ylmethoxy)phenethyl)acrylamide, (E)-3-(3,4-dihydroxyphenyl)-N-(4-(pyridin-2-ylmethoxy)phenethyl)acrylamide, (E)-3-(3,4-dihydroxyphenyl)-N-(4-(2-(dimethylamino)ethoxy)phenethyl)acrylamide, (E)-3-(3,4-dihydroxyphenyl)-N-(4-isobutoxyphenethyl)acrylamide, (E)-3-(3,4-dihydroxyphenyl)-N-(4-(pyridin-4-ylmethoxy)phenethyl)acrylamide, (E)-3-(3,4-dihydroxyphenyl)-N-(4-((4-methoxybenzyl)oxy)phenethyl)acrylamide, (E)-3-(3,4-dihydroxyphenyl)-N-(4-(oxetan-3-ylmethoxy)phenethyl)acrylamide, (E)-3-(3,4-dihydroxyphenyl)-N-(4-((tetrahydro-2H-pyran-2-yl)methoxy)phenethyl)acrylamide, (E)-3-(3,4-dihydroxyphenyl)-N-(4-((tetrahydrofuran-2-yl)methoxy)phenethyl)acrylamide, (E)-3-(3,4 -dihydroxyphenyl)-N-(4-(thiophen-2-yloxy)phenethyl)acrylamide, (E)-3-(3,4-dihydroxyphenyl)-N-(4-(3,3-dimethylbutoxy)phenethyl)acrylamide, (E)-3-(3,4-dihydroxyphenyl)-N-(4-(2-hydroxyethoxy)phenethyl)acrylamide, (E)-N-(4-((1H-tetrazol-5-yl)methoxy)phenethyl)-3-(3,4-dihydroxyphenyl)acrylamide, (E)-3-(3,4-dihydroxyphenyl)-N-(4-((1-methylpyrrolidin-2-yl)methoxy)phenethyl)acrylamide, (E)-2-hydroxy-5-(3-((4-hydroxyphenethyl)amino)-3-oxoprop-1-en-1-yl)phenyl hydrogen carbonate, (E)-3-(4-hydroxy-3-(pyridin-4-yloxy)phenyl)-N-(4-hydroxyphenethyl)acrylamide, (E)-3-(4-hydroxy-3isobutoxyphenyl)-N-(4-hydroxyphenethyl)acrylamide, (E)-3-(3-(4-fluorophenoxy)-4-hydroxyphenyl)-N-(4-hydroxyphenethyl)acrylamide, (E)-3-(3-(cyanomethoxy)-4-hydroxyphenyl)-N-(4-hydroxyphenethyl)acrylamide, (E)-2-(2-hydroxy-4-(3-((4-hydroxyphenethyl)amino)-3-oxoprop-1-en-1-yl)phenoxy)acetic acid, (E)-3-(3-hydroxy-4-(pyridin-4-ylmethoxy)phenyl)-N-(4-hydroxyphenethyl)acrylamide, (E)-3-(4-((4-fluorobenzyl)oxy)-3-hydroxyphenyl)-N-(4-hydroxyphenethyl)acrylamide, (E)-3-(3-hydroxy-4-isobutoxyphenyl)-N-(4-hydroxyphenethyl)acrylamide, (E)-3-(4-(cyanomethoxy)-3-hydroxyphenyl)-N-(4-hydroxyphenethyl)acrylamide, (E)-N-(3-(3,4-dihydroxyphenyl)acryloyl)-N-(4-hydroxyphenethyl)glycine, (E)-3-(3,4-dihydroxyphenyl)-N-(4-hydroxyphenethyl)-N-(pyridin-4-ylmethyl)acrylamide, (E)-3-(3,4-dihydroxyphenyl)-N-(4-hydroxyphenethyl)-N-isobutylacrylamide, (E)-N-(cyanomethyl)-3-(3,4-dihydroxyphenyl)-N-(4-hydroxyphenethyl)acrylamide, 3-(3,4-dihydroxyphenyl)-N-(4-hydroxyphenethyl)propanamide, 3-(3,4-dihydroxyphenyl)-N-(4-(methylsulfonamido)phenethyl)propanamide, or pharmaceutical salts, solvates, and combination of the foregoing.

11. The method of claim 1, wherein the compound of Formula (I) or Formula (II) is in the form of a pharmaceutically acceptable salt.

12. The method of claim 1, wherein the composition of Formula (I) or Formula (II) is in a unit dosage form and is configured for administration between 0.1 and 100 mg/kg of the body weight of the subject of Formula (I) or Formula (II) per administration.

13. The method of claim 1, wherein administering the compound of Formula (I) or Formula (II) reverses hepatic steatosis.

14. The method of claim 1, wherein reversing hepatic steatosis is treating or ameliorating a disease or condition associated with hepatic steatosis.

15. A composition comprising:
one or more compounds selected from a macrolide, a retinide, and a DES1 inhibitor;
a carrier; and
a compound of Formula (I), or an isomer, salt, homodimer, heterodimer, or conjugate thereof: 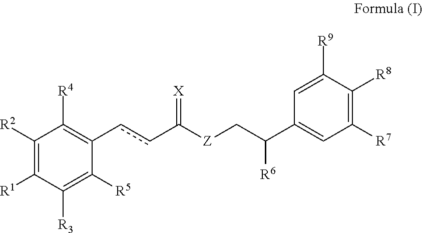
wherein
R¹, R², R³, R⁴, R⁵, R⁶, R⁷, R⁸, and R⁹ are each independently selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆alkenyl, optionally substituted —(O)C₁₋₆ alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkyl C₁₋₁₂heteroaryl; the dashed bond is present or absent;
X is CH₂ or O;
Z is CHR^{a}, NR^{a}, or O; and
R^{a} is selected from hydrogen, deuterium, hydroxyl, halogen, cyano, nitro, optionally substituted amino, optionally substituted C-amido, optionally substituted N-amido, optionally substituted ester, optionally substituted —(O)C₁₋₆alkyl, optionally substituted —(O)C₁₋₆ alkenyl, optionally substituted —(O)C₁₋₆alkynl, optionally substituted, —(O)C₄₋₁₂cycloalkyl, optionally substituted —(O)C₁₋₆alkylC₄₋₁₂cycloalkyl, optionally substituted —(O)C₄₋₁₂heterocyclyl, optionally substituted —(O)C₁₋₆alkylC₄₋₁₂heterocyclyl, optionally substituted —(O)C₄₋₁₂aryl, optionally substituted —(O)C₁₋₆alkylC₅₋₁₂aryl, optionally substituted —(O)C₁₋₁₂heteroaryl, and optionally substituted —(O)C₁₋₆alkylC₁₋₁₂heteroaryl.

16. The composition of claim 15, wherein the retinide is fenretinide, N-(4-hydroxyphenyl) retinamide (4-HPR), 4-oxo-N-(4-hydroxyphenyl) retinamide (4-oxo-HPR), or motretinide.

17. The composition of claim 15, wherein the DES1 inhibitor is selected from N-[(1R,2S)-2-hydroxy-1-hydroxymethyl-2-(2-tridecyl-1-cyclopropenyl)ethyl]octanamide (GT011) and (Z)-4-((5-(4-chlorophenyl)-1,3,4-oxadiazol-2-yl)amino)-N′-hydroxybenzimidamide (B-0027).
